(19) United States
(12) Patent Application Publication
(10) Pub. No.: US 2013/0158614 A1
(43) Pub. Date: Jun. 20, 2013

(54) Pulse Parameters And Electrode Configurations For Reducing Patient Discomfort From Defibrillation

(71) Applicants: Lazaro Salomon Azar, Givatayim (IL); Avi Allon Livnat, Tel Aviv (IL)

(72) Inventors: Lazaro Salomon Azar, Givatayim (IL); Avi Allon Livnat, Tel Aviv (IL)

(73) Assignee: SMARTWAVE MEDICAL LTD, Tel-Aviv (IL)

(21) Appl. No.: 13/806,441

(22) PCT Filed: Jun. 22, 2011
(86) PCT No.: PCT/US11/41411

§ 371 Date: Mar. 7, 2013

Related U.S. Application Data
(60) Provisional application No. 61/400,017, filed on Jul. 22, 2010.
(60) Provisional application No. 61/416,946, filed on Nov. 24, 2010.
(60) Provisional application No. 61/398,665, filed on Jun. 30, 2010.

(30) Foreign Application Priority Data
Jun. 25, 2010 (US) 12/823507

(51) Int. Cl. A61N 1/39

(52) U.S. Cl. CPC A61N 1/3925; A61N 1/39; A61N 1/3937
USPC 607/6; 607/5; 607/8

(57) ABSTRACT

Devices, systems and methods relating to defibrillation and, more specifically, pulse parameters and electrode configurations for reducing patient discomfort are disclosed. Embodiments provide for an implantable defibrillator having an electrode lead system, at least one sensor for sensing a heart condition and emitting a condition signal, a controller in communication with the at least one sensor and configured to determine from the condition signal whether the heart is fibrillating and emitting a command signal if fibrillation is detected and a voltage generator communicating with the controller and the electrode system to communicate at least one defibrillation pulse to the electrode system, wherein the at least one defibrillation pulse includes at least one pulse having a voltage greater than 80 volts and a time duration up to 1000 microseconds.

### CROSS-REFERENCE TO RELATED APPLICATIONS

The present application claims priority to U.S. patent application Ser. No. 12/823,507, filed on Jun. 25, 2010 and entitled “Atrial Defibrillation Using an Implantable Defibrillation System,” which is a continuation-in-part application of International Patent Application No. PCT/US2009/033786, filed on Feb. 11, 2009 and also entitled “Atrial Defibrillation Using an Implantable Defibrillation System,” which claims priority to U.S. Provisional Patent Application No. 61/064,288, filed on Feb. 27, 2008, the disclosures of all of which are incorporated herein by reference in their entirety. The present application also claims priority to U.S. Provisional Patent Application Nos. 61/398,665 filed on Jun. 30, 2010 and entitled “Wave Forms for Atrial Defibrillation,” 61/400,017 filed on Jul. 22, 2010 and entitled “Method for Intra-Cardiac Atrial Defibrillation” and 61/416,946 filed on Nov. 24, 2010 and entitled “Implantable Defibrillation System,” the disclosures of all of which are also incorporated herein by reference in their entirety.

### FIELD

Devices, systems and methods relating to defibrillation and, more specifically, pulse parameters and electrode configurations for reducing patient discomfort are described herein. Some embodiments relate to defibrillating an atrium with one or more high-voltage, short-duration pulses using one or more pairs of electrodes positioned in or around the heart.

### BACKGROUND

Atrial fibrillation (“AF”) is the most common cardiac arrhythmia involving at least one of the right atrium or left atrium. One way to defibrillate an atrium is by delivering electrical defibrillation pulses to the heart at specific times during the cardiac cycle. Systems and devices for delivering these pulses may be external and/or implanted within the body. Atrial defibrillation using an implantable atrial defibrillator generally includes automatically detecting AF and automatically delivering an electrical pulse to the left and/or right atrium. Ventricular fibrillation is also very common. Ventricular defibrillation includes automatically detecting ventricular fibrillation and automatically delivering the electrical pulse to the heart.

Delivering an electrical pulse however may be intolerably painful for a patient and may discourage the use of automatic implantable atrial defibrillators. While delivering an electrical pulse having an energy that is too high may cause pain to a patient, delivering an electrical pulse having an energy that is too low will result in an unsuccessful defibrillation attempt. Accordingly, atrial and/or ventricular defibrillation that is tolerable and effective and/or reduces the discomfort to a patient is desired.

### SUMMARY

In some embodiments described herein, an implantable defibrillator having an electrode lead system with at least one lead, at least one sensor configured to sense a condition of a heart and emit a signal indicative of the condition, a controller in communication with the at least one sensor and being configured to determine from the signal whether the condition of the heart is one of a state of fibrillation and emit a command signal if the condition is one of a state of fibrillation and/or a voltage generator in communication with the controller and the electrode system, the voltage generator being configured to discharge at least one defibrillation pulse to the electrode system after receiving the command signal, wherein the at least one defibrillation pulse includes at least one pulse having a voltage greater than 80 volts and a time duration up to 1000 microseconds. The at least one pulse may be delivered to an atrium and/or a ventricle of the heart and have, according to some embodiments, an electric field strength between 100 and 700 volts per centimeter. The at least one pulse may deliver a total amount of energy to the heart that is less than 2 Joules and/or have a pulse width or time duration between 50 and 600 microseconds, 50 and 1000 microseconds and/or 30 and 100 microseconds. The voltage of the at least one pulse may be between 80 and 3000 volts and/or 600 volts or greater. In some embodiments, the at least one sensor may be an electrode of the electrode lead system.

Some embodiments of the implantable defibrillator may discharge at least one defibrillator pulse that includes at least one pulse having electric field strength between 100 and 700 volts per centimeter, a voltage between 80 and 3000 volts and a time duration between 50 and 1000 microseconds. In some embodiments, the at least one pulse may be synchronized to the patient's cardiac pulse. In some embodiments, the at least one pulse may be synchronized to the patient's cardiac R wave. The at least one pulse, according to some embodiments, may include a first pulse and a second pulse. The first pulse may have a voltage greater than 80 volts and a time duration less than 1000 microseconds and the second pulse may have a voltage greater than 80 volts and a time duration less than 1000 microseconds. In some embodiments, the time duration of the second pulse may be greater than 100 microseconds. The polarity of the first pulse and the polarity of the second pulse may be the same or opposite, depending on the embodiment. The at least one pulse may include a third pulse.

According to some embodiments of the present disclosure, the implantable defibrillator may have a volume less than 15 cubic centimeters. The implantable defibrillator may be configured to be implanted in a location of the heart selected from the group consisting of the pulmonary vein, the subclavian pocket, a branch of the subclavian vein, the left atrium, the right atrium, the right ventricle, the superior vena cava and the inferior vena cava.

Embodiments of the implantable defibrillator may include an electrode lead system with one or more leads positioned in various locations in, on or around the heart. In some embodiments, at least one lead may include at least one electrode positioned in a location of the heart selected from the group consisting of the left atrium, the right atrium, the right ventricle, the coronary sinus of the heart, the pulmonary artery, the apex of the right ventricle and the intra-atrial septum of the heart. Any electrode of the electrode lead system of the present disclosure may be used for discharging electrical pulses or sensing fibrillation. In some embodiments, a lead may be bifurcated and contain a first sub-lead having at least one electrode positioned in the right atrium and a second sub-lead having at least one electrode positioned in at least one of the right ventricle or the left atrium. In some embodiments, the implantable defibrillator may be implanted in the right atrium and the at least one lead may be a single lead having an electrode positioned in at least one of the right ventricle or the left atrium. According to the present disclosure, the implantable defibrillator may act as an electrode positioned within the right atrium.

A lead according to the present disclosure may be a single lead having a first electrode positioned in the right atrium and a second electrode positioned in at least one of the right ventricle or the left atrium. In some embodiments, a lead may be a single lead having a first electrode positioned in the right atrium, a second electrode positioned in the right ventricle and a third electrode positioned in the pulmonary artery. In some embodiments, a lead may be bifurcated and contain a first sub-lead having at least one electrode positioned in the left atrium and a second sub-lead having at least one electrode positioned at apex of the right ventricle.

Embodiments of the electrode lead system of the present disclosure may include a first electrode positioned in the superior vena cava and a second electrode positioned in the left atrium, a first electrode positioned in the superior vena cava and a second electrode positioned in the right ventricle and/or a first electrode positioned in the pulmonary artery and a second electrode positioned in the left atrium.

In some device embodiments, the at least one sensor may include a first sensor and a second sensor in communication with the controller, the first sensor being an electrode for measuring electrical activity of the heart. The second sensor of the at least one sensor may include an electrode for measuring electrical activity of the heart. The second sensor may include a sensing device selected from the group consisting of a microphone, a blood pressure sensor, a thermal sensor, a blood oxygenation sensor, a breathing sensor and an acceleration sensor. In some embodiments, the controller of the implantable defibrillator may be configured to determine a location of fibrillation based on signals received by from the first sensor and the second sensor. The controller may determine a location of fibrillation based on a plurality of electrocardiogram signals and be configured to determine a state of atrial fibrillation based on signals communicated from the first sensor and the second sensor. The controller may be configured to determine a state of atrial fibrillation based on multi-dimensional signal analysis and/or configured to detect a state of ventricle fibrillation and automatically deliver the at least one defibrillation shock when ventricle fibrillation state is detected.

Some embodiments of the electrode lead system of the present disclosure may include a first electrode and a second electrode forming a first pair of electrodes and a third electrode and a fourth electrode forming a second pair of electrodes, wherein a first voltage is applied across the first electrode and the second electrode to form a first electric field and a second voltage is applied across the third electrode and the fourth electrode to form a second electric field. The first electric field may be at an angle relative to the second electric field. The first voltage applied across the first electrode and the second electrode and the second voltage applied across the third electrode and the fourth electrode may not be applied to the heart at the same time. The electrode lead system may include, in some embodiments, a first electrode and a second electrode forming a first pair of electrodes and the first electrode and a third electrode forming a second pair of electrodes, wherein a first voltage is applied across the first electrode and the second electrode to form a first electric field and a second voltage is applied across the first electrode and the third electrode to form a second electric field. In such embodiments, the first electric field may be at an angle relative to the second electric field and/or the first voltage applied across the first electrode and the second electrode and the second voltage applied across the first electrode and the third electrode may not be applied to the heart at the same time.

In some embodiments, the at least one pulse may include:
a monophasic pulse train having at least two pulses with substantially the same polarity, duration and voltage;
a monophasic pulse train having at least a first pulse and a second pulse with substantially the same polarity and voltage, wherein the second pulse has a greater voltage than the first pulse;
a biphasic pulse train having two pulses with the substantially the same polarity, duration and voltage, wherein the biphasic pulse train may have at least a first pulse and a second pulse with substantially the same polarity and voltage, wherein the second pulse has a greater voltage than the first pulse;
a triphasic pulse train having at least three pulses with alternating polarity and substantially the same duration, wherein the initial voltage of each consecution pulse is approximately equal to or slightly less than the final voltage of the preceding pulse;
a monophasic pulse train having at least three pulses, wherein the initial voltage of each consecution pulse is approximately equal to or slightly less than the final voltage of the preceding pulse;
a monophasic pulse train having at least four pulses with substantially the same polarity, voltage and duration;
a monophasic pulse train having at least three pulses with substantially the same polarity and different voltage and duration;
a triphasic pulse train having at least three pulses with alternating polarity and substantially the same voltage and duration;
a triphasic pulse train having at least three pulses with alternating polarity and substantially the same duration, wherein the voltage of each consecutive pulse is larger than the voltage of the preceding pulse;
a monophasic pulse train having at least three pulses with substantially the same polarity, voltage and duration, wherein the dwell time between each pulse is substantially larger than the duration of each pulse;
a biphasic pulse train having at least a first pulse, a second pulse and a third pulse with different voltages and durations, wherein the first pulse and the second pulse are consecutive and have substantially the same polarity, the polarity of the first pulse and the second pulse being different than the polarity of the third pulse;
a pulse train having at least a first pulse of less than 2 Joules and used to measure tissue impedance; and/or
a triphasic pulse train having at least three pulses with alternating polarity and different voltage and duration.

In some device embodiments, the electrode lead system may include a first single lead contain an electrode positioned in the inter-atrial septum and a second single lead containing an electrode positioned in the coronary vein.

Some embodiments of the present disclosure contemplate heart defibrillation systems. Such systems may include a defibrillator configured to be implanted in a patient. The defibrillator may include an electrode lead system having at least one lead, at least one sensor configured to sense a condition of a heart and emit a signal indicative of the condition, a controller in communication with the at least one sensor and that is configured to determine from the signal whether the condition of the heart is one of a state of fibrillation and emit a command signal if the condition is one of a state of fibrillation, a voltage generator in communication with the controller and the electrode system and that is configured to discharge at least one defibrillation pulse to the electrode system after receiving the command signal, wherein the at least one defibrillation pulse includes at least one pulse having a voltage greater than 80 volts and a time duration up to 1000 microseconds and a communication device disposed outside of the patient configured to communicate with the defibrillator. In some embodiments, the communication device may include notification circuitry configured to notify the patient that fibrillation was detected. The notification circuitry may be configured to notify the patient that fibrillation was detected and to instruct the patient to be prepared for an defibrillation shock, instruct the patient to seek medical treatment in a medical center and/or to notify the patient of a worsening cardiac condition.

In some embodiments, the communication device may be configured to initiate an atrial defibrillation shock and/or configured to notify a medical facility of a cardiac condition of the patient. The communication device may include location determination circuitry configured to determine a location of the patient and is configured to communicate the determined location to a medical center. The communication device may be configured for bi-directional communication with the implantable defibrillator over a short range wireless communication link and/or configured for bi-directional communication with the medical center over a long-range wireless communication link. The long-range wireless communication link may be a cellular communication link and the communication device may be a mobile phone. The message communicated over the long-range communication link may be a message selected from the group consisting of a synthesized voice announcement, a pre-recorded voice announcement, a short message service, a multimedia message service and electronic mail.

Some embodiments of the present disclosure contemplate methods for defibrillating a heart with an implantable defibrillator. Methods according to the disclosed subject matter may include detecting a condition of fibrillation within the heart, configuring at least one electrical pulse parameter to define an electrical pulse having a voltage between 80 and 3000 volts and a duration between 30 and 1000 microseconds, generating a first electrical pulse in accordance with the at least one electrical pulse parameter and discharging the first electrical pulse to the heart using an electrode lead system having at least one pair of electrodes positioned in or around the heart. The discharging of the first electrical pulse may include generating an electric field strength of between 100 and 700 volts per centimeter across the at least one pair of electrodes. Some method embodiments may include transmitting a fibrillation message to a medical center when the atrium in the heart fibrillates and/or determining a location of the implantable heart defibrillator using location determination circuitry, the location being included in a fibrillation message that enables the medical center to determine the location of the implantable defibrillation system. Some method embodiments may include delivering a drug to the heart using the implantable heart defibrillator before discharging the first electrical pulse to the atrium of the heart. Some method embodiments may include activating a notification circuitry configured to notify a patient of the first electrical pulse before discharging the first electrical pulse to the atrium of the heart.

Some method embodiments of the present disclosure may reduce pain while defibrillating an atrium of a human heart by delivering at least one pulse to the atrium having a voltage greater than 600 volts and a time duration between 50 and 600 microseconds.

Some method embodiments of the present disclosure may reduce pain associated with defibrillating a ventricle of a human heart by detecting a condition of ventricular fibrillation within the heart using an implantable defibrillator, configuring at least one electrical pulse parameter to define an electrical pulse having a voltage between 80 and 3000 volts and a duration of 50 to 1000 microseconds, generating a first electrical pulse in accordance with the at least one electrical pulse parameter and discharging the first electrical pulse from the implantable defibrillator to the heart using an electrode lead system having at least one pair of electrodes positioned in or around the heart, wherein a total amount of energy delivered by the first electrical pulse is less than 2 Joules, which is an amount of energy that is lower than that delivered by conventional defibrillators. The first electrical pulse may be monophasic or biphasic. In some embodiments, the implantable defibrillator itself may act as an electrode of the electrode lead system. The advantages of such a defibrillator from energy standpoint is longer life of an affiliated power source (e.g., a battery). Patient discomfort is an issue when misfires happen and is also an important attribute to such a defibrillator.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1A shows a block diagram of an implantable atrial defibrillator according to some embodiments of the present disclosure.

FIG. 1B shows a block diagram of an implantable atrial defibrillator according to some embodiments of the present disclosure.

FIG. 1C shows a block diagram of an implantable atrial defibrillator according to some embodiments of the present disclosure.

FIG. 1D shows an embodiment of the controller according to some embodiments of the present disclosure.

FIG. 2 shows a defibrillation system according to some embodiments of the present disclosure.

FIG. 3A shows a chart of defibrillation threshold voltage versus pulse duration of a defibrillation shock according to some embodiments of the present disclosure.

FIG. 3B shows a chart of muscle motion versus pulse duration of the defibrillation shock according to some embodiments of the present disclosure.

FIG. 3C shows a chart of delivered energy versus pulse duration of the defibrillation shock according to some embodiments of the present disclosure.

FIG. 4A shows a single monophasic defibrillation pulse according to some embodiments of the present disclosure.

FIG. 4B shows a single biphasic defibrillation pulse according to some embodiments of the present disclosure.

FIGS. 5A and 5B show implantable defibrillation systems with bifurcated main leads according to some embodiments of the present disclosure.

FIG. 6 shows an embodiment of an electrode lead system with a single electrode on a single main lead according to some embodiments of the present disclosure.

FIGS. 7A and 7B show implantable defibrillation systems with a single electrode on the main lead according to some embodiments of the present disclosure.

FIG. 8 shows one embodiment of an electrode lead system with a plurality of electrodes on the main lead according to some embodiments of the present disclosure.

FIGS. 9A-9D show implantable defibrillation systems with a plurality of electrodes on the main lead according to some embodiments of the present disclosure.

FIGS. 10A-10E show exemplary locations for the discharge electrode and receive electrode according to some embodiments of the present disclosure.

FIG. 11 shows a flow diagram of a method for performing atrial defibrillation according to some embodiments of the present disclosure.

FIG. 12A graphically depicts why defibrillation efficiency may be increased by using more than one electrode pair to deliver defibrillation pulses according to some embodiments of the present disclosure.

FIGS. 12B and 12C show cross-sectional views of a torso of a patient containing implanted defibrillation electrodes showing defibrillating electric fields according to some embodiments of the present disclosure.

FIGS. 13A-13O defibrillation pulse train waveforms produced according to some embodiments of an implantable defibrillator of the present disclosure.

FIG. 14 shows one possible embodiment of two cardioverting electrodes located near the pulmonary veins orifices and each having a lead according to some embodiments of the present disclosure.

Like reference symbols in the various drawings indicate like elements.

### DETAILED DESCRIPTION

The subject matter described herein relates to defibrillation of the heart using an implantable defibrillation system and is not limited in its application to the details set forth in the following disclosure or exemplified by the illustrative embodiments. The subject matter is capable of other embodiments and of being practiced or carried out in various ways. Moreover, features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or as suitable in any other described embodiment of the present disclosure. Certain features described in the context of various embodiments are not to be considered essential features of those embodiments, unless the embodiment is inoperative without those elements.

A significant amount of pain may be felt by a patient during defibrillation of the heart. Evidence suggests that muscle movement during a defibrillation shock is directly proportional to the amount of pain felt by a patient. Studies conducted by Applicants have shown that the amount of muscle movement in the chest region of a patient may be lessened by delivering defibrillation pulses that have higher amplitudes, but shorter pulse widths than those produced by known defibrillation systems, which in turn reduces pain. For example, in one study conducted by Applicants, several pigs were fitted with a defibrillation system configured to deliver high-voltage, short-duration pulses. Such systems are described in more detail herein. The pigs were anesthetized and fitted with accelerometers configured to measure muscle movement around the heart while defibrillation shocks of varying amplitudes and pulse widths were delivered.

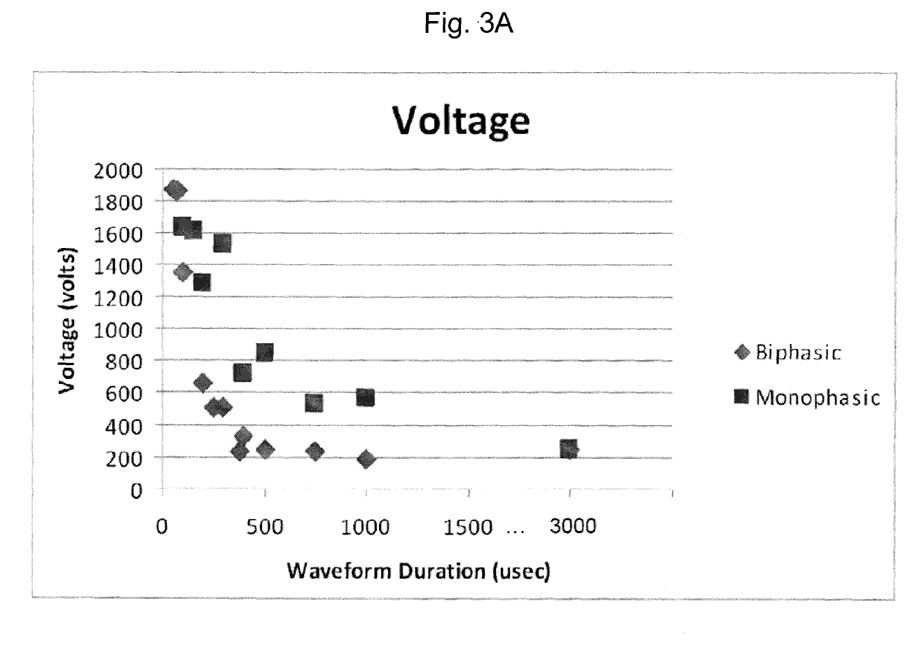

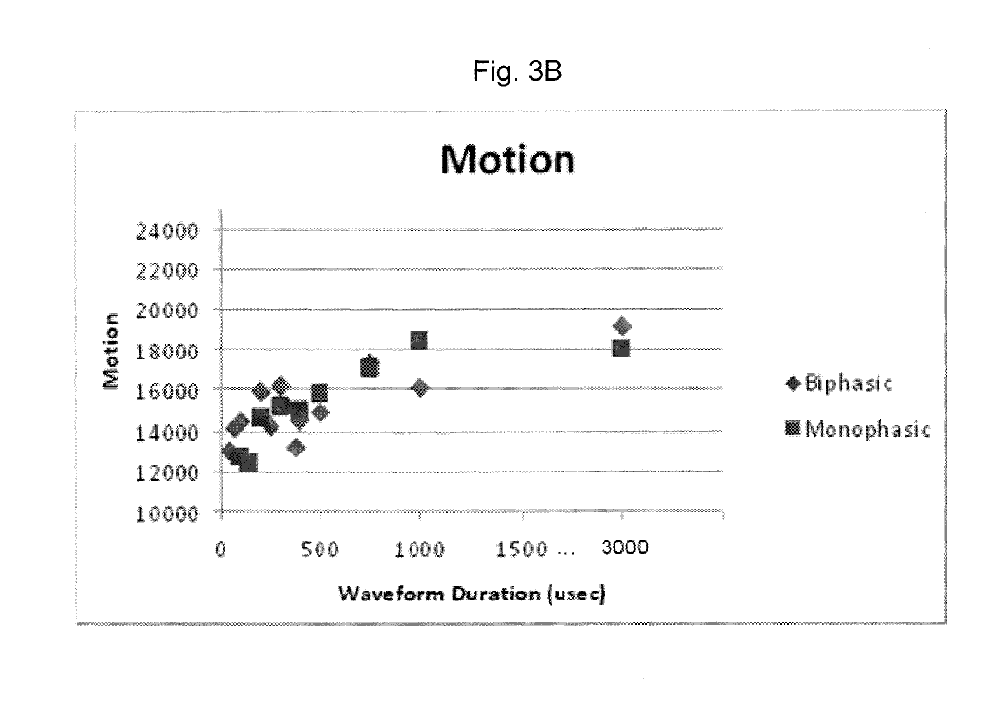

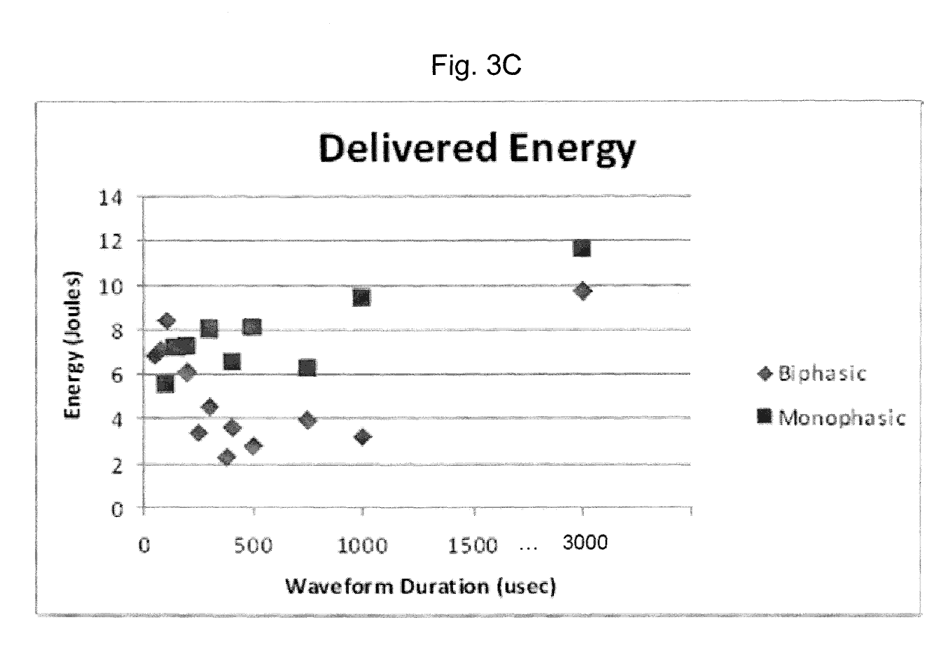

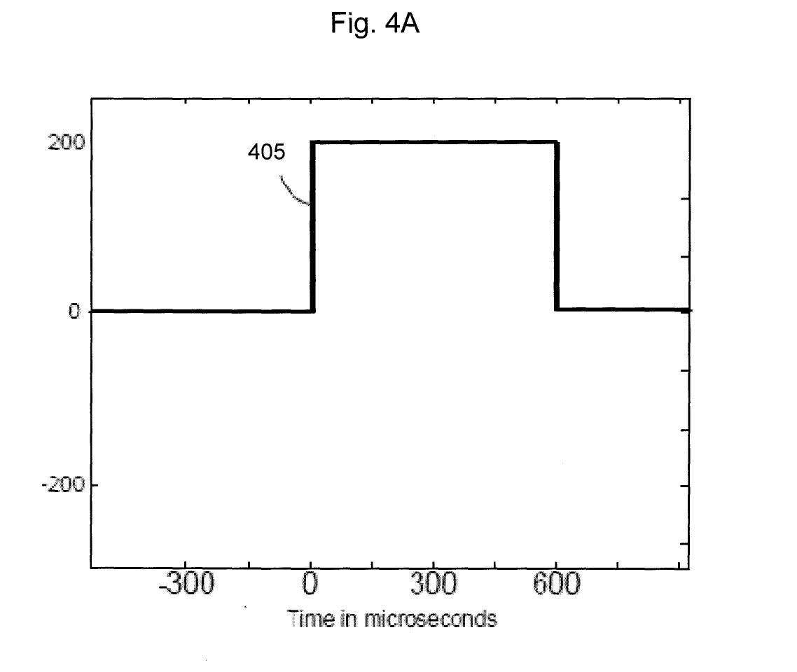

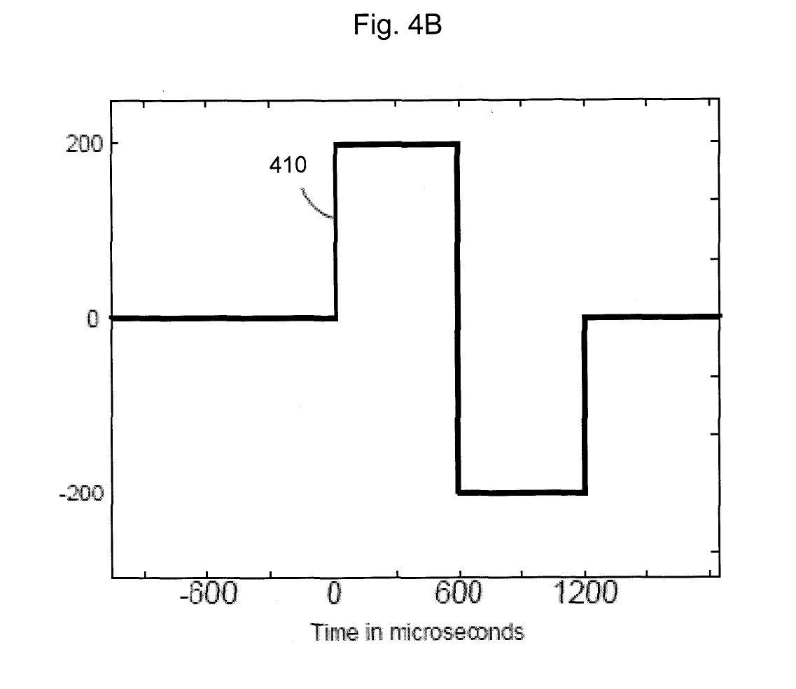

The results are shown in FIGS. 3A-3C. FIG. 3A shows the defibrillation threshold voltage versus the pulse duration of a defibrillation shock capable of stopping fibrillation of the heart. The x and y axes represent the pulse duration in microseconds and the voltage of the defibrillation shock, respectively. FIG. 3A shows the voltages and pulse widths for defibrillation shocks that successfully stopped fibrillation. These pulses include biphasic and monophasic pulses and are described in more detail below with reference to FIGS. 4A and 4B. As shown in FIG. 3A, as the pulse width of the defibrillation pulse decreases, the amount of voltage needed to defibrillate the heart increases. The voltage required for a biphasic pulse remains relatively constant for pulse widths above about 200/200 microseconds and increases rapidly for shorter pulse widths. All pulse width values associated with biphasic pulses specified herein correspond to the pulse width of each phase of the biphasic pulse width. The pulse width may or may not be denoted with a “/” symbol to denote the pulse widths of both phases. The voltage required for a monophasic pulse gradually increases as the pulse width narrows to about 500 microseconds and then increases rapidly for shorter pulse widths. Overall, the amount of voltage needed for a biphasic pulse is less than that of a monophasic pulse.

FIG. 3B shows muscle movement around the heart versus the pulse duration of the defibrillation shock. The X and Y axes represent the pulse width in microseconds and the relative motion of the muscles around the heart, respectively. As shown in FIG. 3B, the muscle movement remains substantially constant for pulse widths greater than 1000 microseconds. Unexpectedly, however, the amount of muscle movement decreases significantly for shorter pulse widths. In particular, for monophasic pulses the muscle movement decreases for pulse widths below 600 microseconds, and for biphasic pulses, the muscle movement decreases for pulse widths below 1000 microseconds. It is believed that a reduction in muscle movement will result in a corresponding reduction in pain felt by a defibrillation shock. Accordingly, the decrease in muscle movement associated with the shorter pulses is believed to result in less pain felt during a defibrillation shock applied with the devices, systems and methods of the present disclosure. However, as shown in FIG. 3A, the defibrillation threshold voltage required for defibrillating the heart increases as the pulse width decreases

FIG. 3C shows the amount of energy versus the pulse duration of the defibrillation shock. The X and Y axes represent the pulse width in microseconds and the energy of the defibrillation shock, respectively. As shown in FIG. 3C, the amount of energy required for a biphasic pulse decreases as the pulse width decreases to about 200 microseconds. Unexpectedly, the amount of defibrillation energy increases sharply for shorter pulse widths. For monophasic pulses, the amount of defibrillation energy decreases gradually as the pulse width decreases. Overall, the amount of defibrillation energy for biphasic pulses is less than that for a monophasic pulse. It can be shown that the amount of energy delivered for either waveform is lower than the amount of energy delivered in known defibrillation systems. In addition to minimizing pain, another advantage of lower energy is that it enables the miniaturization of the implantable device, thus enabling the production of defibrillation systems smaller than known systems. Such smaller systems may, for example, be implanted transcutaneously. Moreover, the lower energy level extends the usable life of a power source (e.g., a battery) and/or enables the use of a smaller power source.

Accordingly, the pulse width may be decreased to reduce the amount of pain felt as a result of a defibrillation shock. However, for biphasic pulse widths shorter than about 50/50 microseconds and monophasic pulses shorter than around 50 microseconds the amount of defibrillation voltage may be such that a defibrillation shock may cause tissue damage. The pulse width may be maintained in an optimum range. In some embodiments of the present disclosure, the range for a biphasic pulse may be between 50/50 and 600/600 microseconds and the defibrillation voltage may be 80 volts or greater. The range for a monophasic pulse may be between 50 and 600 microseconds and the defibrillation voltage may be 80 volts or greater

Embodiments of the present disclosure may be directed to reducing the pain and/or discomfort of atrial and/or ventricular defibrillation by defibrillating the heart using an electrical pulse with a field strength of 100-700 volts/cm. In some embodiments, the pulse width may be 30-50 microseconds. In some embodiments, the electrical pulse may have a voltage of at least 600 volts. In some embodiments, the electrical pulse may have a voltage greater than 80 volts. The amount of voltage needed to perform defibrillation may be increased or decreased depending on various physiological factors. For example, as noted above, the data shown in FIGS. 3A and 3B are the results of tests performed on pigs. These tests show that for pulses between 50 and 600 microseconds, a defibrillation voltage of 80 volts may suffice. A human heart, on the other hand, may require a different defibrillation voltage. The defibrillation voltage may be higher or lower depending on the individual anatomy and the specific physiological response of the person. Therefore, the defibrillation voltage may be adjusted accordingly.

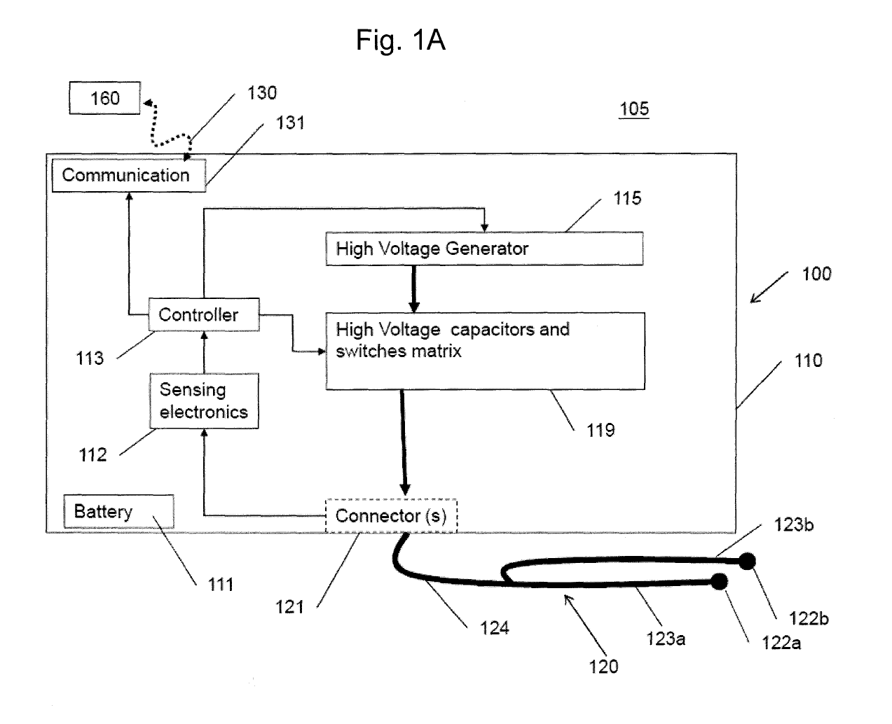

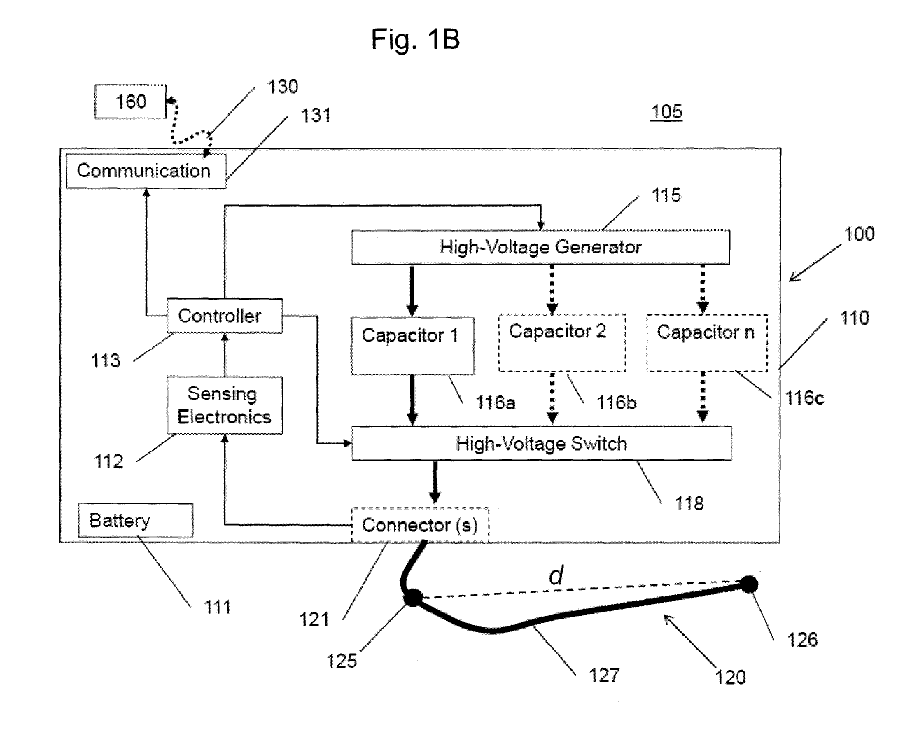

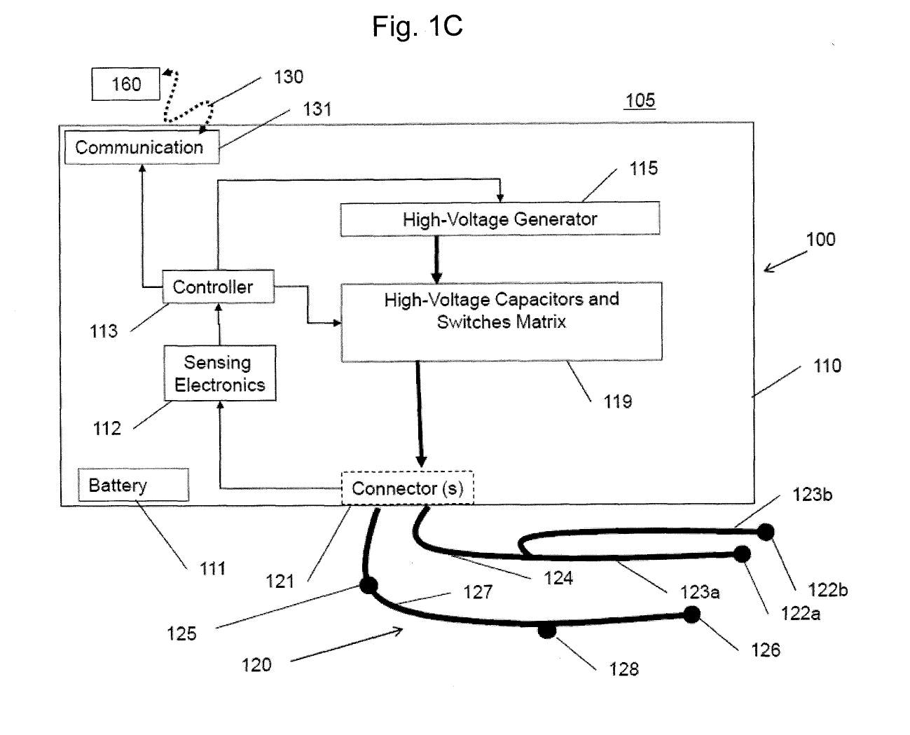

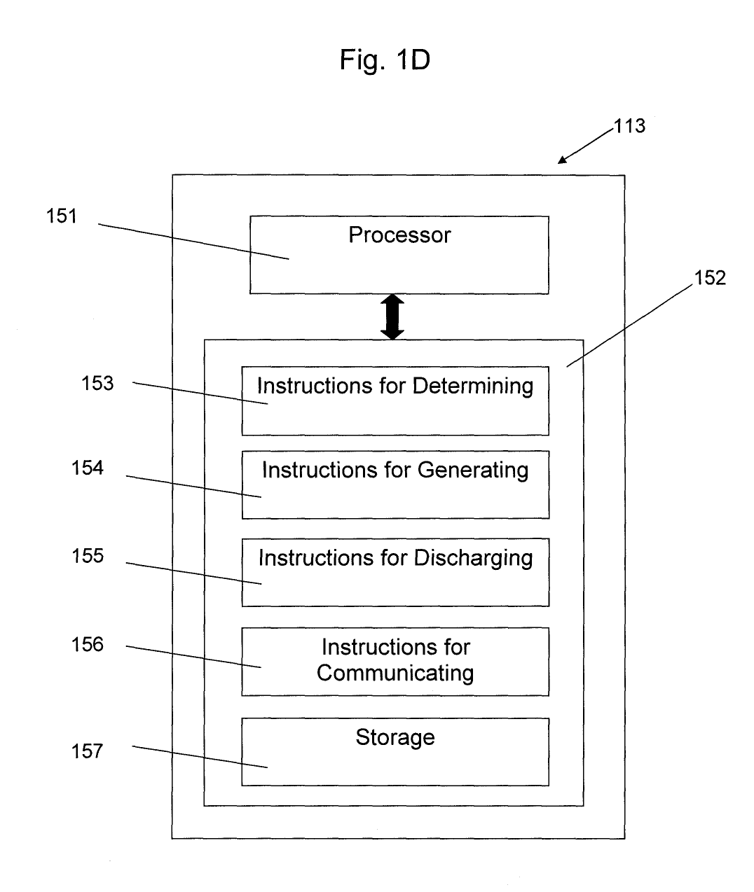

FIG. 1A shows a block diagram of an implantable defibrillation system (105) that includes an implantable defibrillator (100) and a communication device (160), according to some embodiments of the present disclosure. The implantable defibrillator (100) may be configured to defibrillate the atria and/or ventricles of the heart of a patient. The implantable defibrillator (100) includes a defibrillator body (110) and an electrode lead system (120). The defibrillator body (110) may be coupled to the electrode lead system (120). The phrase “coupled to” as used herein means directly connected to or indirectly connected through one or more intermediate components. Such intermediate components may include both hardware and software-based components. The internal construction of the implantable defibrillator (100) may vary depending upon the embodiment and, in some embodiments, may be an internal construction that is known in the art. Example configurations of the implantable defibrillator (100) are provided in International Publication No. WO2009/108502 to Livnat et al., filed on Feb. 11, 2009 and entitled “Atrial Defibrillation Using an Implantable Defibrillation System,” the disclosure of which is incorporated herein by reference in its entirety.

The implantable defibrillation system (105) discussed herein may be implemented in any of a number of configurations, such as an implantable miniature atrial defibrillator, implantable heart defibrillator, defibrillation implant, an implantable cardioverter defibrillator, a pacemaker system, a ventricular defibrillation system, other system used for atrial defibrillation or any combination thereof. In some embodiments, the implantable defibrillation system (105) may be a combination of a pacemaker system and an atrial defibrillator. In some embodiments, the implantable defibrillation system (105) may be a combination atrial/ventricular defibrillation system that includes a pacemaker system.

The implantable defibrillator (100) may include communication circuitry (131) (e.g., a transceiver) capable of wirelessly communicating with external communication device (160) using a communication link (130). The communication link (130) may have short-range and/or long-range capabilities. The communication link (130) may be an ultrasonic link communicating with an external device in contact with a patient's body. In some embodiments, the communication link (130) may be a short-range radio frequency (“RF”) communication link and may use a proprietary protocol for communicating with an interface device. In some embodiments, the communication link (130) may use a common protocol, such as Bluetooth technology or wireless fidelity (“Wi-Fi”), wherein the external device may include mobile devices (i.e., portable devices), such as, for example, a mobile phone, media player, smartphone, Personal Digital Assistant (PDA), other handheld computing devices and the like.

The defibrillator body (110) of implantable defibrillator (100) may be a bio-compatible housing or enclosure, canister, conductive enclosure, atrial defibrillator housing, other defibrillation body or a combination thereof. The defibrillator body (110) may or may not be constructed from a conducting material. For example, all, some or none of the defibrillator body (110) may include or be coated with metal, such as gold or titanium. The defibrillator body (110) may enclose one, some or all of the components depicted in FIGS. 1A-1C and/or described herein. For example, a sensing electronic module (112) may be disposed outside the defibrillator body (110) and electrically connected to a controller (113) enclosed within the defibrillator body (110). In some embodiments, as discussed below, the defibrillator body (110) itself may serve as a sensing and/or shocking electrode.

The defibrillator body (110) may be sized to be implanted in the heart or surrounding regions. The defibrillator body (110) may be sized to be implanted within the pulmonary vein, the subclavian pocket, the right atrium, a branch of the subclavian vein, the vena cava or a different location. In some embodiments, the defibrillator body (110) may be sized to be positioned outside, around or adjacent to the pulmonary vein, the subclavian pocket, the right atrium or a branch of the subclavian vein. The shape of defibrillator body (110) may be a box, rectangular volume or other shaped volume that encloses the system components disclosed herein. The size (e.g., length, height and/or volume) of the defibrillator body (110) may depend on the size of the enclosed components. In some embodiments, as shown in FIGS. 1A-1C, the defibrillator body (110) may enclose at least the controller (113), a high-voltage generator (115) and a high-voltage capacitors and switches matrix module (119). The high-voltage capacitors of the module (119) may be sized to store low energy according to some embodiments. The phrase “low energy” as used herein may include without limitation energy in or around the range of 0.1-2 joules. The phrase “high energy” as used herein may include without limitation energy in or around the range of greater than 2 joules. A low-energy capacitor may have a smaller size than a high-energy capacitor. In some embodiments, the defibrillator body (110) may be less than 20 cubic centimeters and, in some instances, 5-15 cubic centimeters.

The implantable defibrillator (100) may contain at least one power source (111). The power source (111) may be a battery, power pack or other device that provides power to one or more of the other components of the implantable defibrillator (100). The power source (111) may be coupled to the sensing electronics module (112), the controller (113), the high-voltage generator (115), the high-voltage capacitors and switches matrix module (119) or any combination thereof. In some embodiments, the power source (111) may be rechargeable. In some embodiments, the power source (111) needed for charging an atrial defibrillation capacitor may be smaller than a power source (111) for charging a ventricular defibrillation capacitor; however, because the load caused by the sensing electronics module (112) may remain similar, the proportional size savings for the power source (111) may be smaller. Periodic recharging of the power source (111) may reduce the size of the battery to substantially the size needed to produce defibrillation shocks. In a device intended to deliver only a few defibrillation shocks before being replaced or recharged, the size of the power source (111) may be greatly reduced. In some embodiments, a first power source (111) may be used for storing energy needed for defibrillation while a second power (111) may power the sensing electronics module (112) and/or the controller (113). The power source (111) may be inductively rechargeable, such that the power source (111) does not need to be removed from the patient to be recharged.

In some embodiments, the implantable defibrillator (100) may also contain electronic circuitry for sensing cardiac activity, processing the sensed activity to determine whether the activity is normal or indicative of a fibrillation state, and delivering one or more high-voltage defibrillation pulses. In some embodiments, the implantable defibrillator (100) and the electronic circuitry therein may be configured to differentiate between atrial and ventricular fibrillations and respond accordingly based on whether the atria or ventricles of the heart are fibrillating.

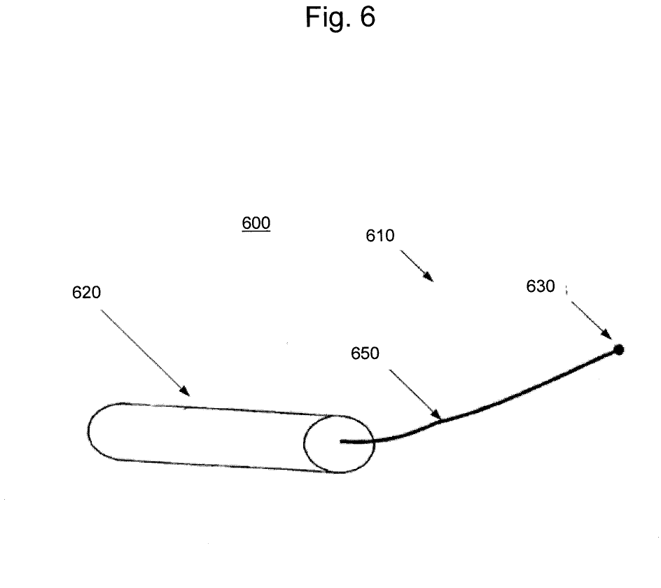

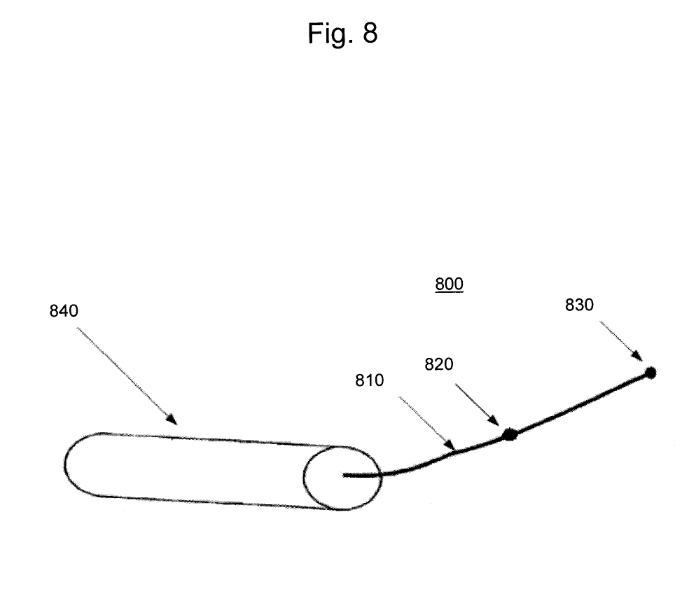

Some embodiments of the defibrillator body (110) may include at least one electrical connector (121) connected to the electrode lead system (120). In some embodiments, the electrode lead system (120) may have a main lead. The main lead may have two or more sub-leads. For example, the electrode lead system (120) shown in FIG. 1A contains a bifurcated main lead (124) having two sub-leads (123a) and (123b). The sub-leads (123a) and (123b) may contain electrodes (122a) and (122b), respectively, as shown in FIG. 1A. According to the present disclosure, the number of main leads, sub-leads and electrodes positioned on the main leads and/or sub-leads is unlimited and will vary depending upon the desired configuration. In some embodiments, as shown in FIG. 1B, the electrode lead system (120) may include a single main lead (127) having a single discharge electrode (125) and a single receiving electrode (126). In such embodiments, the implantable defibrillator (100) may generate one or more electrical pulses that are discharged from electrode (125) and received at electrode (126). In some embodiments, the electrode lead system (120) may include a bifurcated main lead (124) having two sub-leads (123a) and (123b) that contain electrodes (122a) and (122b), respectively, and a single main lead (127) having a single discharge electrode (125) and a single receiving electrode (126). In some embodiments, the single main lead (127) may contain one or more central electrodes (128) positioned between the discharge electrode (125) and receiving electrode (126). Similar electrode lead system configurations are also shown in FIGS. 6 and 8 and discussed in more detail herein.

An electrode, a sensor or a combination thereof may be disposed anywhere along the bifurcated main lead (124), single main lead (127) and/or any sub-leads (e.g., 123a, 123b). The bifurcated main lead (124), single main lead (127) and/or any sub-leads (e.g., 123a and 123b) may be any suitable length. For example, sub-lead (123a) may be longer than sub-lead (123b) and/or the bifurcated main lead (124) and the sub-leads (123a) and (123b) may, in total, be longer than the single main lead (127). The leads and/or sub-leads of the electrode lead system (120) embodiments of the present disclosure may include, without limitation, a wire, rod, flexible arm, clamp or other device for positioning any electrodes thereon within, on, adjacent to or around the heart of a patient. The leads and/or sub-leads of the electrode lead system (120) embodiments of the present disclosure may be electrically conductive, such that electrical signals may be transmitted along the leads and/or sub-leads to one or more electrodes positioned thereon. The electrical signals may include cardiac functioning signals, electrical pulses and/or other communication signals.

The connector (121) may include a mating connector, a lead connector or other connector for coupling the electrode lead system (120) to the defibrillator body (110). For example, as shown in FIGS. 1A-1C, the connector (121) may be a mating connector that connects the bifurcated main lead (124) and/or single main lead (127) to the high-voltage capacitors and switches matrix module (119) in the defibrillator body (110). In some embodiments, the electrode lead system (120) may be permanently attached to the defibrillator body (110).

Embodiments of the present disclosure provide for numerous configurations of electrode placement in, on and/or around the heart of a patient. For example, some embodiments of the implantable defibrillator (100) may position one or more electrodes in left and/or right atrium for pacing the heart, in addition to one or more electrodes used for atrial defibrillation. In some embodiments, one or more electrodes may be positioned in the right ventricle and used for electrocardiogram (ECG) sensing and delivering one or more ventricular defibrillation pulses or pulse trains. In some embodiments, the defibrillator body (110), or parts thereof, may be used as an electrode. In some embodiments, the communication circuitry (131) may use one or more leads and/or sub-leads of the electrode lead system (120) as an antenna for radiofrequency (RF) communication. Some embodiments of the implantable defibrillator (100) may include a dedicated antenna, for example a coil, loop or dipole antenna, located within or outside the defibrillator body (110). One or more electrodes on the leads and/or sub-leads may be used for sensing ECG signals for monitoring the cardiac activity of a patient implanted with the implantable defibrillator (100). In some embodiments, one or more of the same electrodes may be used for both sensing ECG data and delivering defibrillation pulses or cardiac pacing. In some embodiments, at least one electrode may be dedicated to sensing ECG signals.

According to some embodiments of the present disclosure, the sensing electronic module (112) of the implantable defibrillator (100) may have one or more sensing electrodes configured to condition (e.g., amplify and/or filter) ECG signals and monitor cardiac activity and other bodily functions. In some embodiments, the implantable defibrillator (100) may include one or more thermal sensors to monitor patient body temperature, blood oxygenation sensors, microphones to monitor sound emitted from the heart and the respiratory system, breathing sensors (e.g., capacitive sensors or sensors sensing the bending of a lead or sub-lead due to breathing) and/or other sensors known in the art, including without limitation, pressure sensors, blood pressure sensors, acceleration sensors or any other sensors for receiving cardiac functioning signals. In some embodiments, sensor electronics may include an Analog-to-Digital Converter (ADC).

The sensing electronic module (112) may be disposed within or outside of the defibrillator body (110). The sensing electronic module (112) may be a separate component or integrated with another component of the implantable defibrillator (100), such as the electrode lead system (120). In some embodiments, the sensing electronic module (112) may be configured with an electrode that is connected to the electrical connector (121) to function as both an electrode for delivering an electrical pulse and as a component of the sensing electronic module (112) by, for example, providing cardiac functioning signals (e.g., ECG signals) to the controller (113). In some embodiments, the sensing electronic module (112) may be connected to a pressure meter disposed in a vein (e.g., vena cava) or in an atrium. In some embodiments, a plurality of the same or different sensing electronic modules (112) may be used.

In some embodiments, the sensing electronic module (112) may receive cardiac functioning signals. Receiving the cardiac functioning signals may include, without limitation, sensing, detecting, determining, monitoring or any combination thereof. For example, an accelerometer may sense the motion of the heart. The sensing electronic modules (112) may provide the cardiac function signals to the controller (113). The controller (113) may use the cardiac function signals to determine whether an atrium and/or ventricle is in a state of fibrillation or experiencing some other abnormal heart rhythm condition. In some embodiments, the sensing electronic module (112) may include a processor that uses the cardiac function signals to determine whether the atrium and/or ventricle is in a state of fibrillation and emit a condition signal indicating that an atrium and/or ventricle is fibrillating.

Embodiments of the implantable defibrillator (100) may include the controller (113) for performing signal conditioning and analysis. The controller (113) may receive data indicative of cardiac activity from the sensing electronic module (112) and other optional sensors and/or may receive commands and data from the communication circuitry (131). The controller (113) may determine the state of the cardiac activity based on ECG signals and other sensor data and control the pulse-generating circuitry to produce one or more defibrillation pulses when appropriate. In some embodiments, the sensing electronic module (112) and the controller (113) may be used to determine whether an atrium is fibrillating. The sensing electronic module (112) may detect a set of measurements, for example, using a plurality of sensing electrodes and/or other sensors (e.g., acoustic). The sensing electronic module (112) may transmit the set of measurements to the controller (113), which calculates the probability that atrial fibrillation exists and may issue commands based upon that decision.

FIG. 1B shows a block diagram of an embodiment of the implantable defibrillator (100) according to some embodiments of the present disclosure. The implantable defibrillator (100) shown in FIG. 1B does not include the high-voltage capacitors and switches matrix module (119) shown in FIGS. 1A and 1C. Instead, embodiments of the present disclosure according to FIG. 1B may include at least one high-voltage capacitor (116a) coupled to the high-voltage generator (115) and capable of being charged to a desired high voltage by the high-voltage generator (115). The implantable defibrillator (100) shown in FIG. 1B may also include a high-voltage switch (118) that discharges voltage stored in the high-voltage capacitor (116a) into one or more electrodes coupled to the implantable defibrillator (100).

As shown in FIG. 1B, the discharge electrode (125) on the single main lead (127) may receive voltage from high-voltage capacitor (116a) via the connector (121). This voltage may also be transmitted along the single main lead (127) to receiving lead (126). The lead (127) couples the discharge electrode (125) to the receiving electrode (126). In some embodiments, the controller (113) may include the discharge electrode (125) and/or the defibrillator body (110) may enclose the discharge electrode (125). In some embodiments, the high-voltage switch (118) may control pulse duration. The implantable defibrillator (100) shown in FIG. 1B may also include one or more additional high-voltage capacitors (116b) and (116c) for generating a train of pulses. The pulses in the train may have the same or opposite polarity and/or different voltage and pulse duration. Some embodiments of the implantable defibrillator (100) may have a patient notification element, such as a vibrator or buzzer, to alert a patient when atrial and/or ventricular fibrillation has been detected.

FIG. 1C shows an embodiment of an implantable defibrillator (100) that is substantially similar to the embodiment of the implantable defibrillator (100) depicted in FIG. 1A, but is coupled to a configuration of the electrode lead system (120) that is different from that which is shown in FIG. 1A. More specifically, in addition to the bifurcated main lead (124) having two sub-leads (123a) and (123b), FIG. 1C shows the electrode lead system (120) also having the single main lead (127) having a single discharge electrode (125) and a single receiving electrode (126), wherein the implantable defibrillator (100) may generate one or more electrical pulses that are discharged from electrode (125) and received at electrode (126).

As shown in FIG. 1D, some embodiments of the controller (113) may include a processor (151) and memory (152). The controller (113) may be a computer, processing system or a circuit for instructing and controlling the components of the implantable defibrillator (100). The controller (113) may be coupled to the sensing electronic module (112), the high-voltage generator (115), the high-voltage capacitors and switches matrix module (119), the electrical connector (121) and/or the power source (111). The controller (113) may control the generation of electrical pulses. In some embodiments, the electrical pulse may have a field strength of 100-700 volts/cm and/or a discharge voltage of 600 volts or greater. In some embodiments, the electrical pulse may have a discharge voltage of 80 volts or greater. The controller (113) may also control the discharge of the electrical pulses, such that one or more of the discharged electrical pulses have a time duration of up to 600 microseconds and, in some embodiments, up to 1000 microseconds.

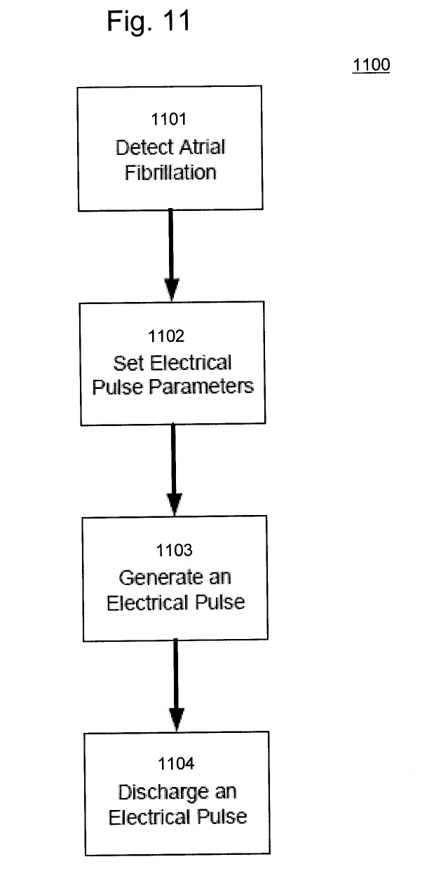

The processor (151) may be a general processor, digital signal processor, application-specific integrated circuit, field programmable gate array, analog circuit, digital circuit, combinations thereof or other now known or later developed processor. The processor (151) may be a single device or a combination of devices, and may be associated with a network or distributed processing system. Any of various processing strategies may be used, including without limitation, multi-processing, multi-tasking, parallel processing or the like. Processing may be local or remote. In some embodiments, a communication device may be used to transmit signals received by the processor (151) to a remote processor, which is operable to process the received signals. The processor (151) may be responsive to instructions stored as part of software, hardware, integrated circuits, firmware, micro-code or the like. The processor (151) may be operable to perform one or more of the steps illustrated in FIG. 11.

In some embodiments, the processor (151) may be operable to determine whether an atrium or ventricle is fibrillating. Determining whether the heart is fibrillating may include receiving one or more cardiac functioning signals from one or more sensors in the sensing electronic module (112). The processor (151) may analyze the one or more cardiac functioning signals to determine whether an atrium and/or ventricle is fibrillating. In some embodiments, the processor (151) may compare a spatial point that represents the current state of an atrium and/or ventricle, to a multi-dimensional space. The multi-dimensional space may indicate a fibrillation space and a non-fibrillation space. When the spatial point is in the non-fibrillation space, an atrium and/or ventricle is not fibrillating. When the spatial point is in the fibrillation space, an atrium and/or ventricle is fibrillating.

In some embodiments, the processor (151) may be operable to determine electrical pulse parameters for defibrillation of the heart. The electrical pulse parameters include a discharge voltage and an electrical pulse time duration. The electrical pulse parameters define an electrical pulse at the time of discharge. Once it is determined that the heart is fibrillating, the processor (151) may generate and deliver a command signal containing the electrical pulse parameters to one or more of the components of the implantable defibrillator (100), including without limitation the high-voltage capacitors and switches matrix module (119) and/or the high-voltage generator (115). In some embodiments, the electrical pulse parameters may define a discharge voltage of at least 80 volts, which may be determined as a function of the field strength of the electrical pulse between one or more discharges electrodes and one or more receiving electrodes. In some embodiments, the field strength may be proportional to the voltage difference between a discharge electrode and a receiving electrode and inversely proportional to the distance between the two. Accordingly, the discharge voltage may be determined as a function of the distance and the desired field strength between a discharge electrode and receiving electrode. In some embodiments, the distance between a discharge electrode and a receiving electrode may be the shortest distance between them, as shown in FIG. 1B where distance, d, is the shortest distance between discharge electrode (125) and receiving electrode (126). In some embodiments, the distance between the discharge electrode (125) to the receiving electrode (126) may be distance along the main lead (127). According to some embodiments of the present disclosure, the distance between the discharge electrode to the receiving electrode may be in or around the range of 2.0-12.0 centimeters or any other suitable distance.

In some embodiments, the processor (151) may be operable to activate the high-voltage generator (115) to charge one or more of the high-voltage capacitors in the high-voltage capacitors and switches matrix module (119) according to one or more the electrical pulse parameters. The high-voltage generator (115) may be activated with a command signal from the controller (113). In some embodiments, the high-voltage generator (115) may be controlled to charge one or more the high-voltage capacitors to a voltage of at least 80 volts and, in some embodiments, a voltage of 600 volts or greater, 1000 volts or greater, 1300 volts or greater and/or up to 3000 volts. In some embodiments, one or more high-voltage capacitors may be charged to a voltage of 600-1000 volts, 1000-1300 volts and/or 1300-3000 volts. According to the present disclosure, higher voltages may be combined with shorter defibrillation pulse widths to effectively defibrillate the heart. In some embodiments, electrical pulses provided by the high-voltage generator (115) may have a field strength of 100-700 volts/cm and may have a low energy so as to reduce the size of the components in the implantable defibrillator (100). An electrical pulse with a field strength of 100-700 volts/cm may also ensure that the electrical pulse will defibrillate the heart without injuring the patient's heart. In some cases, a field strength of less than 100 volts/cm could be ineffective in defibrillating the heart, and a field strength of greater than 700 volts/cm could seriously injure the patient's heart. In some embodiments, the electrical pulse provided by the high-voltage generator (115) may have a field strength of 100-300 volts/cm or a field strength of 300-700 volts/cm. In some embodiments, the high-voltage capacitors and switches matrix module (119) may include a capacitor bank that is operable to provide one or more electrical pulses to the switches matrix of the module (119).

In some embodiments, the controller (113) may be operable to activate a high-voltage switch, as depicted by reference numeral (118) in FIG. 1B. Activating the high-voltage switch (118) may include discharging energy from one or more high-voltage capacitors, such as high-voltage capacitors (116a), (116b) and/or (116c) to the electrical connector (121) and the electrode lead system (120). The time duration of one or more electrical pulses provided by the high-voltage capacitors may be controlled by the controller (113). The duration of each electrical pulse may be up to 600 microseconds, including without limitation, the time durations (i.e., pulse widths) of 30-100 microseconds, 30-50 microseconds, 50-70 microseconds and/or 70-100 microseconds.

The pulse shape of the electrical pulse may also be controlled by the controller (113). As shown in FIGS. 4A and 4B, electrical pulses may be monophasic and/or biphasic. A monophasic pulse (405) is shown in FIG. 4A. A biphasic pulse (410) is shown in FIG. 4B. The amplitude of the pulses (405) and (410) may remain substantially constant within the pulse width of the pulses (405) and (410). In some embodiments, the duty cycle of biphasic pulse (410) may be about 50% and may differ from the amplitude of each phase of the pulse. The area under the curve defined by the voltage and pulse width in microseconds of the positive phase may equal the area under the curve defined by the voltage and pulse width in microseconds of the negative phase, such that the net electrical charge to the heart after a defibrillation shock is substantially zero, as shown in FIG. 4B.

Tables 1 and 2 below illustrate exemplary voltage and pulse width combinations that may be utilized to defibrillate the heart according to embodiments of the present disclosure.

TABLE 1
Voltage/Duration Ranges

| | 100-1000 μsec | <100 μsec |
|---|---|---|
| 80-800 Volts | x | |
| 800+ Volts | | x |

TABLE 2
Voltage/Duration Ranges

| | 30-50 μsec | 50-70 μsec | 70-100 μsec |
|---|---|---|---|
| 600-1000 Volts | x | x | x |
| 1000-1300 Volts | x | x | x |
| 1300+ Volts | x | x | x |

Table 3 below illustrates exemplary field strength and pulse width combinations that may be utilized to defibrillate the heart according to embodiments of the present disclosure.

TABLE 3
Field Strength Ranges

| | 30-50 μsec | 50-70 μsec | 70-100 μsec |
|---|---|---|---|
| 100-300 volts/cm | x | x | x |
| 300-700 volts/cm | x | x | x |

The processor (151) may be operable to control the discharge of an electrical pulse train. The electrical pulse train may include one or more electrical pulses. The electrical pulses in the electrical pulse train may have the same or different pulse widths, discharge voltages and/or field strength values. In some embodiments, an electrical pulse train may include a first electrical pulse and a second electrical pulse. The first electrical pulse and the second electrical pulse may have discharge voltages that are at least 80 volts and pulse widths of at least 50-600 microseconds. In some embodiments, the first electrical pulse may have a discharge voltage of at least 1000 volts and a pulse width of 30-100 microseconds, and the second electrical pulse may have a discharge voltage of less than 600 volts, a pulse duration less than 30 microseconds or greater than 100 microseconds or a combination thereof.

In some embodiments, the controller (113) may have a memory (152) that includes, without limitation, computer readable storage media. The computer readable storage media may include volatile and/or non-volatile memory. The memory (152) may be a single device or a combination of devices. The memory (152) may be adjacent to, part of, networked with and/or remote from the processor (151). The memory (152) may store information, signals or other data. As shown in FIG. 1D, the memory (152) may include storage (157), which is used to store information, signals or other data. For example, the storage (157) may be used to store ECG data, monitored vital signs, patient events, a log of device activity, status events and operation parameters. Other information or data relating to or not related to atrial defibrillation may be stored in the storage (157).

In some embodiments, the memory (152) may store instructions for the processor (151). The processor (151) may be programmed with and execute the instructions. The functions, acts, methods or tasks illustrated in the figures or described herein may performed by the processor (151) executing instructions stored in the memory (152). The functions, acts, methods or tasks may be independent of the particular type of instructions set, storage media, processor or processing strategy and may be performed by software, hardware, integrated circuits, firm ware, micro-code and the like, operating alone or in combination. The instructions may be configured for implementing the processes, techniques, methods or acts described herein.

As shown in FIG. 1D, the memory (152) may include instructions for determining (153), instructions for generating (154), instructions for discharging (155) and instructions for communicating (156). The memory (152) may include additional, different or fewer instructions. The instructions for determining (153) may relate to determining fibrillation of the heart of a patient and be executed to determine whether an atrium and/or ventricle is fibrillating. In some embodiments, the instructions for determining (153) may be executed to process signals, which may be provided by sensors, to determine whether an atrium and/or ventricle is fibrillating. The instructions for generating (154) may relate to generating one or more electrical pulses and be executed to generate one or more electrical pulses having voltages of 80 volts or greater, pulse widths of up to 1000 microseconds and/or field strengths of 100-700 volts/cm. Accordingly, the instructions for generating (154) may be executed to command the high-voltage generator (115) of the implantable defibrillator (100) to charge one or more high-voltage capacitors to discharge high-voltage pulses. The instructions for discharging (155) may relate to discharging high-voltage pulses and be executed to deliver one or more electrical pulses having voltages of 80 volts or greater, pulse widths of up to 1000 microseconds and/or field strengths of 100-700 volts/cm to one or more electrodes. The instructions for communicating (156) may relate to communicating with a communication device and be executed to communicate with a communication device, such as communication device (160), in, around and/or outside of the defibrillation system (105).

In some embodiments, the high-voltage generator (115) of the implantable defibrillator (100) may charge one or more high-voltage capacitors of the high-voltage capacitors and switches matrix module (119) as shown in FIGS. 1A and 1C or, in some embodiments, one or more high-voltage capacitors (116a-116c) as shown in FIG. 1B. High-voltage capacitors of the implantable defibrillator (100) may store energy to provide electrical pulses to one or more electrodes in accordance with electrical pulse parameters. In some embodiments, the high-voltage generator (115) may charge one or more high-voltage capacitors to provide electrical pulse, which has a voltage of at least 80 volts may be provided to a discharge electrodes. In some embodiments, the implantable defibrillator (100) may include the high-voltage generator (115), a single high-voltage capacitor and a high-voltage switch. In some embodiments, the implantable defibrillator (100) may include a capacitor bank, where more than one capacitor is used to store energy to provide for selective discharge of electrical pulses in the form of a pulse train.

A high-voltage switch (118), as shown in FIG. 1B and according to some embodiments of the present disclosure, may be activated by the controller (113) or an activation circuit to discharge energy from one or more high-voltage capacitors (116a-116c). The controller (113) may activate the high-voltage switch (118) when a high-voltage capacitor (e.g., 116a) has been charged to a specific voltage. In some embodiments, the high voltage switch (118) may include an activation circuit that activates the high-voltage switch (118) when one or more high-voltage capacitor(s) (e.g., 116a-116c) have been charged to one or more specific voltages. The activation circuit may include a gas discharge tube, silicone-controlled rectifier and/or light-activated, silicon-controlled rectifier. Once activated, the high voltage switch (118) may allow a high-voltage capacitor to discharge and send a defibrillation shock to one or more electrodes of the electrode lead system (120) of the implantable defibrillator (100). In some embodiments, a high-voltage switch (118) may reverse the high-voltage polarity during a defibrillation pulse and/or safely discharge one or more high-voltage capacitors (e.g., 116a-116c) if fibrillation stops while the capacitor(s) are charging.

Some embodiments of the implantable defibrillator (100) may include communication circuitry (131) that enables the implantable defibrillator (100) to communicate with an externally-located communication device (160) as shown in FIGS. 1A-1C. The communication device (160) may be a device, such as a cellular phone or smartphone, configured to communicate information over relatively long distances. In some embodiments, the communication circuitry (131) may be configured to wirelessly communicate with the communication device (160) 10 meters or more away from the implantable defibrillator (100). The communication circuitry (131) may implement a Bluetooth protocol or any other protocol that enables communications with the communication device (160). In operation, the communication circuitry (131) may be utilized to communicate information, such as trigger information, warning signals, distance information, voltage difference information, software updates, fibrillation messages and/or other messages to and from the implantable defibrillator (100). In some embodiments, the communication device (160) may communicate with the implantable defibrillator (100) via a wired connection. The communication between the implantable defibrillator (100) and the communication device (160) may be unidirectional or bidirectional and/or may be half duplex or full duplex.

The implantable defibrillator (100) may also be configured to initiate the communication of information to the communication device (160) and/or respond to requests from the communication device (160). The communication may be used to program, set up and/or monitor the implantable defibrillator (100), as well as query or interrogate the implantable defibrillator (100) for alarm data, to verify functionality and to download stored information, such as patient heart activity and device activity data from the implantable defibrillator (100).

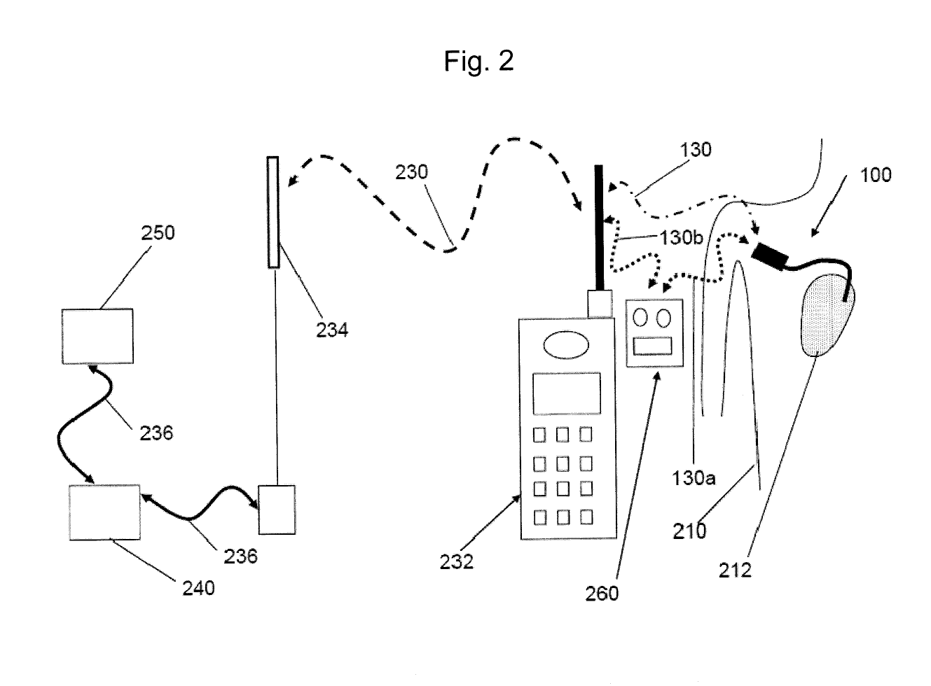

FIG. 2 shows a defibrillation system (200) using an embodiment of the implantable defibrillator (100) according to the subject matter of the present disclosure. In some embodiments of the system (200), the implantable defibrillator (100) may be implanted in a patient (210). One or more electrodes may be positioned in, on or around the heart (212) of the patient (210). The system (200) may include an external communication device (232), an interface device (260) and a server (240), all of which may be in wireless communication with one another. In some embodiments, the implantable defibrillator (100) may communicate directly with the server (240) or via the external communication device (232) and/or the interface device (260) to, for example, transmit data to the server (240) relating to a possible fibrillating state.

The implantable defibrillator (100) of the system (200) may communicate with the external communication device (232). The communication between the implantable defibrillator (100) and the external communication device (232) may be short-range and/or long-range communication. The external communication device (232) may be configured as a two-way communicator capable of transmitting and receiving both data and voice information or, alternatively, the external communication device (232) may be configured to transmit and receive only data or only voice information. In some embodiments, the external communication device (232) may include one or more user inputs, such as a keypad, touch screen, scroll wheel or microphone. Some embodiments of the external communication device (232) may have one or more user outputs, such as a display screen, speaker, vibrating mechanism and/or light-emitting component (e.g., a light-emitting diode). The external communication device (232) may also include a GPS receiver for determining the location of the external communication device (232). The external communication device (232) may be a cellular phone, a smartphone or any other handheld computing device. In some embodiments, external communication device (232) may also be a satellite communication device.

In some embodiments, the implantable defibrillator (100) may communicate with the external communication device (232) via the communication link (130), as shown in FIG. 2. The implantable defibrillator (100) may, in some embodiments, communicate with the external communication device (232) via an interface device (260). In some embodiments, the interface device (260) may be an application embedded within external communication device (232). In some embodiments, the external communication device (232) and/or the interface device (260) may be embedded within the implantable defibrillator (100) itself, either as software and/or hardware components of the implantable defibrillator (100). Other embodiments of the present disclosure contemplate the interface device (260) as a separate component in wireless communication with implantable defibrillator (100), server (240) and/or external communication device (232). In such embodiments, the interface device (260) may be any shape or size. The interface device (260) may be miniature for discreet placement in or around the heart (212) of the patient (210). The interface device (260), in some embodiments, may be used primarily for providing an interface between the implantable defibrillator (100) and the external communication device (232) and, thus, may contain no user inputs or outputs. In other embodiments, the interface device (260) may communicate directly with the server (240). The interface device (260) may include user inputs, such as switches or buttons, and user outputs, such as a display screen, speaker(s) and/or vibrating mechanism. Communication between the implantable defibrillator (100) and the external communication device (232) via the interface device (260) may involve using short-range channels. As shown in FIG. 2, the implantable defibrillator (100) may communicate with the interface device (260) via a short-range channel (130a) and the interface device (260) may communicate with the external communication device (232) via a short-range channel (130b). In some embodiments, the channels connecting the implantable defibrillator (100), interface device (260) and external communication device (232) may be long-range channels or a combination of short-range and long-range channels.

FIG. 2 also shows that the external communication device (232) may communicate with the server (240) via a long-range communication channel (230). For example, the external communication device (232) may be a mobile phone that communicates with a base station (234) over a long-range communication channel (230), such as a cellular RF channel, and connect to the server (240) over a channel (236). The channel (236) may be a land line, cellular line or other communication channel, such as the Internet. In some embodiments, the external communication device (232) may be a satellite communication device capable of communicating with the server (240) from anywhere around the world. The server (240) may constitute a medical center, hospital and the like, as well as any computers, hospital equipment and human personnel located at any such facility.

In some embodiments, the server (240) may communicate with a rescue team (250) (e.g., a medical team, paramedics and/or an ambulance) over the channel (236) (e.g., land or cellular lines) and direct the rescue team (250) to the location of the patient (210). In some embodiments, the external communication device (232) may communicate directly with the rescue team (250). The communicated message may be a fibrillation message that indicates that the patient's heart is fibrillating and that an electrical pulse has been or will be discharged automatically. The fibrillation message may include other information, such as information that identifies the patient, the age and gender of the patient and/or other information that enables a medical technician to determine the best course of action for dealing with the patient's condition. Other messages may be communicated. For example, a phone message with a synthesized voice announcement and/or a pre-recorded voice announcement, a short message service (SMS) text message, a session initiation protocol message, a multimedia messaging service (MMS) message, an electronic mail (e-mail) message or other type of audio or data message may be communicated. Alternatively, the message from the external communication device (232) may be a code recognizable by the server (240) that triggers generation and transmission of such voice, SMS and other messages by the server (240).

In some embodiments, a medical technician may communicate a command message to the implantable defibrillator (100) to initiate a defibrillation pulse via the communication device (160). For example, a fibrillation message may be communicated to medical staff at a hospital indicating that a patient is experiencing fibrillation. Once the patient arrives at the hospital, a medical attendant may, via the communication device (160), communicate a command message to the implantable defibrillator (100) to command the implantable defibrillator (100) to generate a discharge pulse. This advantageously allows the medical attendant to supervise the defibrillation of the patient's heart. In some embodiments, additional, different or fewer components may be provided in the implantable defibrillator (100). For example, the implantable defibrillator (100) may include a microphone, notification circuitry, a location device or a combination thereof. The microphone may be utilized for receiving information from an ultrasonic transducer in contact with the patient's body.

The notification circuitry may correspond to a vibration device or acoustical device, configured to warn a patient about an impending discharge before discharging the electrical pulse. For example, the notification circuitry may generate an alarm to warn a patient that fibrillation was detected. The warning may be provided one or more seconds before the discharge. Accordingly, the patient may have time to prepare for the electrical pulse, for example, by pulling off to the side of the road when driving. In one embodiment, the warning may be provided such that the patient knows to go to a hospital or medical facility and has time to make it to the hospital or medical facility. Once at the hospital or medical facility, a discharge pulse may be automatically or manually discharged, such that the defibrillation is conducted under the supervision of a medical expert. In this example, the detection and alarm are automatic; whereas, the discharge of the defibrillation electrical pulse or pulse train is manual.

The location device is configured to determine a geographic location of a patient. For example, the location device may include global positioning circuitry. The location device is operable to determine a location of the implantable defibrillator (100). The location may be transmitted to a server (240), such as a computer network at a hospital or medical facility. For example, the communication device (160) may transmit a fibrillation message to the server (240). The fibrillation message may include the patient's location, since the implantable defibrillator (100) is implanted in the patient.

In some implementations, the microphone, notification device and/or the location device are located within the defibrillator body (110). In other implementations, the microphone, notification device, and/or the location device are located external to the patient. For example, the various devices may be located within an external communication (see reference numeral 232 in FIG. 2). In some embodiments, the functions that utilize the microphone, notification device and/or the location device may be implemented by an application configured to operate on a mobile device equipped with such hardware. For example, a cellular telephone may correspond to a smartphone equipped with GPS hardware, a microphone, a speaker and/or any other hardware describe herein. One or more applications for implementing the functions above associated with the microphone, notification device and/or the location device may be stored on and executed by a processor of the mobile device (e.g., cellular telephone). Placement of these components outside of the patient enables the size of the defibrillator body (110) to be reduced. In some embodiments, implantable defibrillator (100) may include a hook device operable to connect one or more electrodes of the implantable defibrillator (100) to a wall of the heart.

According to the subject matter of the present disclosure, electrodes of the embodiments described herein may be used for sensing fibrillation (e.g., with the sensing electronics module (112)) and/or for shocking the heart (e.g., with the high-voltage generator (115), high-voltage capacitors and switches matrix module (119), high-voltage capacitors (116a-116c) and/or high-voltage switch (118)). The electrodes may be positioned in, on or around the various parts of the heart, including without limitation, the right atrium (e.g., near the atrioventricular (AV) node); the left atrium (e.g., in the coronary sinus via the right atrium and the coronary sinus ostium), the right ventricle near the pulmonary valve (e.g., via the right atrium and the tricuspid valve), the pulmonary artery near the pulmonary valve (e.g., via the right atrium and the tricuspid valve, through the right ventricle and through the pulmonary valve), the apex of the right ventricle or any combination thereof. In some embodiments, the apex of the right ventricle may be used only for sensing fibrillations. In one embodiment, the defibrillator body (110) itself may be used as a sensing and/or shocking electrode. In some embodiments, at least two electrodes may be used for sensing and/or shocking, wherein any combination of two or more electrodes may be combined to provide for shocking and/or and sensing functionality.

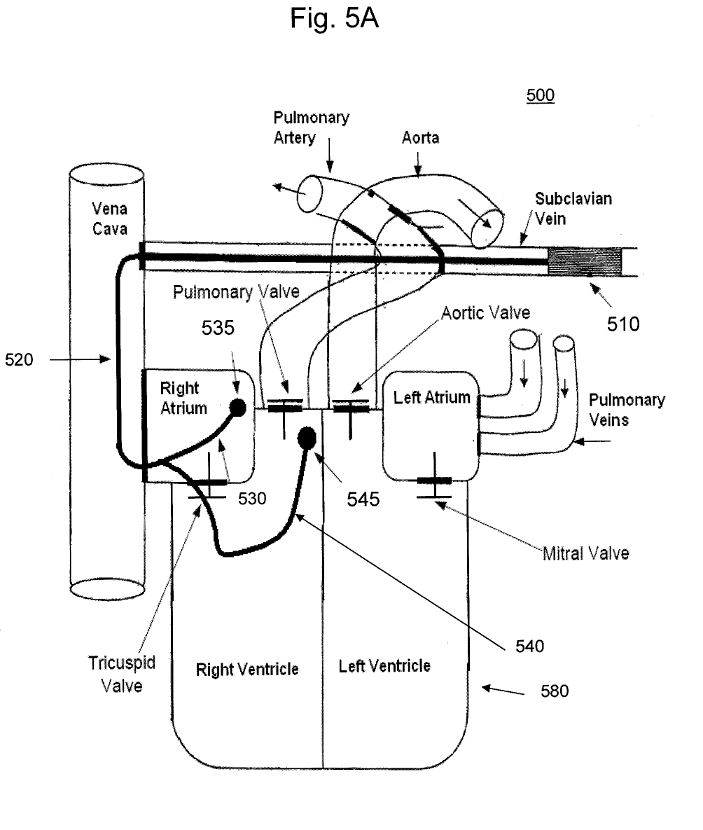

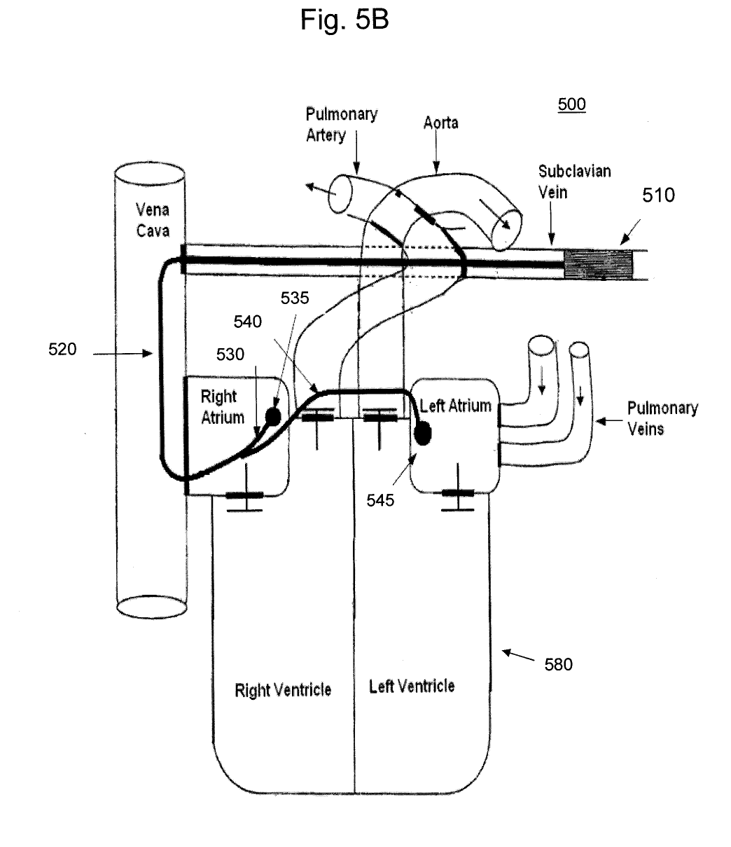

FIGS. 1A-1C, 6 and 8 show example configurations of electrode lead systems according to the subject matter of the present disclosure. FIGS. 5A, 5B, 7A, 7B, 9A-9D and 10A-10E show example configurations of electrode placement in, on and around the heart according to the subject matter of the present disclosure. These examples are not limiting and other embodiments of the electrode lead system (120) and/or other electrode placement configurations may be used for defibrillating the heart.

FIGS. 5A and 5B illustrate an electrode lead system (500) having a defibrillator (510) and a bifurcated lead (520) for defibrillating a heart (580). FIG. 5A shows the defibrillator (510) being located in a side branch of the subclavian vein and the bifurcated lead (520) traversing the subclavian vein, into the vena cava and entering into the heart (580) from the vena cava. In some embodiments, the defibrillator (510) and/or the bifurcated lead (520) may be positioned solely in the vena cava. The bifurcated lead (520) may have two or more sub leads. FIG. 5A depicts an embodiment having a first sub-lead (530) containing an electrode (535) and a second sub-lead (540) containing an electrode (545). The electrode (535) of the first sub-lead (530) is positioned in the right atrium and the electrode (545) of the second sub-lead (540) is positioned in the right ventricle. In some embodiments, the second sub-lead (540) may enter the right ventricle through the tricuspid valve. In some embodiments, the distal end of the first sub-lead (530) and/or second sub-lead (540) may be attached to a wall of the right atrium (e.g., in various locations including the AV node or the sinoatrial (SA) node) and/or a wall of the right ventricle, such that the electrode (535) and/or electrode (545) contact the walls of the right atrium and/or right ventricle. FIG. 5B shows a configuration similar that shown in FIG. 5A, with the exception of the electrode (545) of the second sub-lead (540) being positioned in the left atrium, rather than the right ventricle. In some embodiments, the second sub-lead (540) may be inserted into the left atrium through the coronary sinus and near the pulmonary valve.

FIG. 6 shows a configuration of an electrode lead system (600) according to some embodiments of the present disclosure. The system (600) may have a receiving electrode (630) disposed at one end of an electrode lead (610). The electrode lead (610) may include a main lead (650). An electrode, a sensor and/or a combination thereof may be disposed on the main lead (650). The main lead (650) may be coupled to a defibrillator (620) for defibrillating an atrium and/or ventricle of a heart. In some embodiments, the defibrillator (620) may be connected to the system (600) via a special connector (not shown). The defibrillator (620) may be displaced from the system (600), for example, subcutaneously implanted, similar to current pacemakers and/or defibrillators.

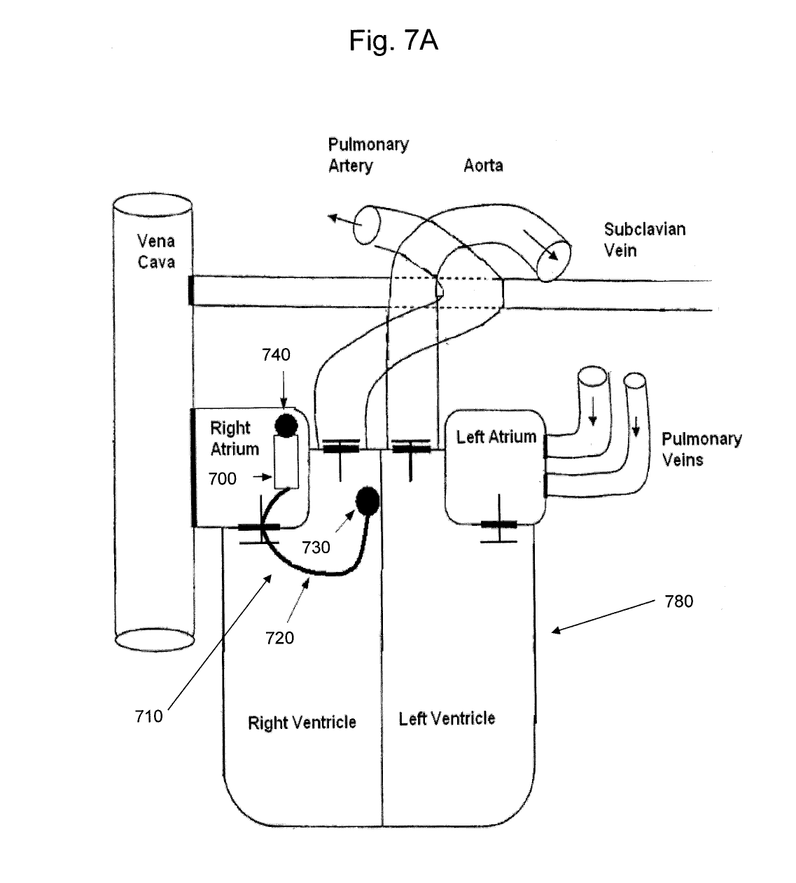

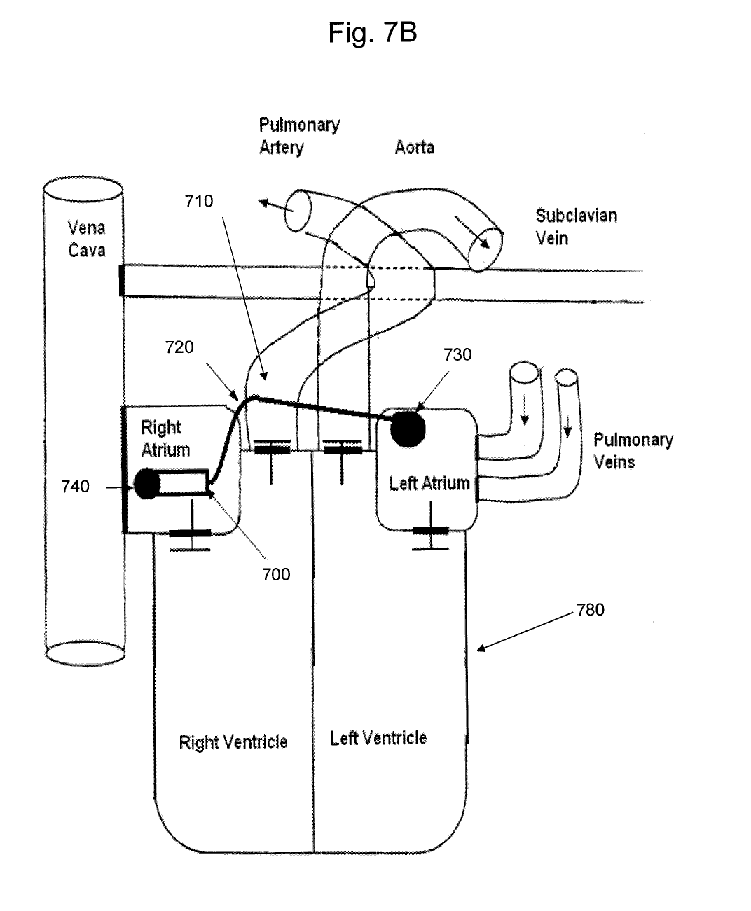

FIGS. 7A and 7B show a defibrillator (700) implanted within the right atrium of a heart (780). In some embodiments, the defibrillator (700) may act as or include an electrode (740), as well as an electrode lead system (710) having an electrode lead (720) containing an electrode (730) disposed at a distal end of the electrode lead (720). The defibrillator (700) may be positioned in the right atrium and, in some embodiments, attached to a wall of the right atrium, whereby electrode (740) directly contacts the right atrium wall. The electrode lead (720) may enter the right ventricle through the tricuspid valve and be anchored to a wall of the right ventricle, such that the electrode (730) directly contacts the right ventricle wall. FIG. 7B shows a configuration similar that shown in FIG. 7A, with the exception of the electrode (730) being positioned in the left atrium, rather than the right ventricle. In some embodiments, the electrode lead (720) may be inserted into the left atrium through the coronary sinus and near the pulmonary valve.

FIG. 8 shows an electrode lead system (800) having a pigtail lead configuration. A plurality of electrodes, sensors or a combination thereof may be disposed on a main lead (810). In some embodiments, a central electrode (820) may be disposed on the main lead (810) between a receiving electrode (830) and a defibrillator (840). The central electrode (820) may be a sensing electrode (e.g., part of the sensing electronics modules 112) or a discharge electrode for discharging electrical pulses. In some embodiments, the defibrillator (840) may be connected to the system (800) via a special connector (not shown). The defibrillator (840) may be displaced from the system (800), for example, subcutaneously implanted, similar to current pacemakers and/or defibrillators.

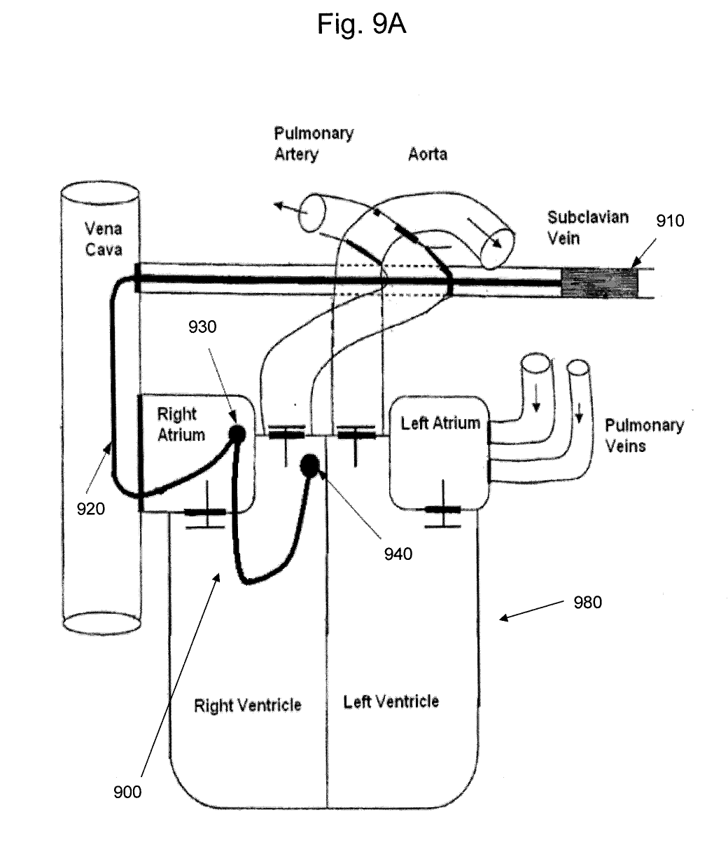

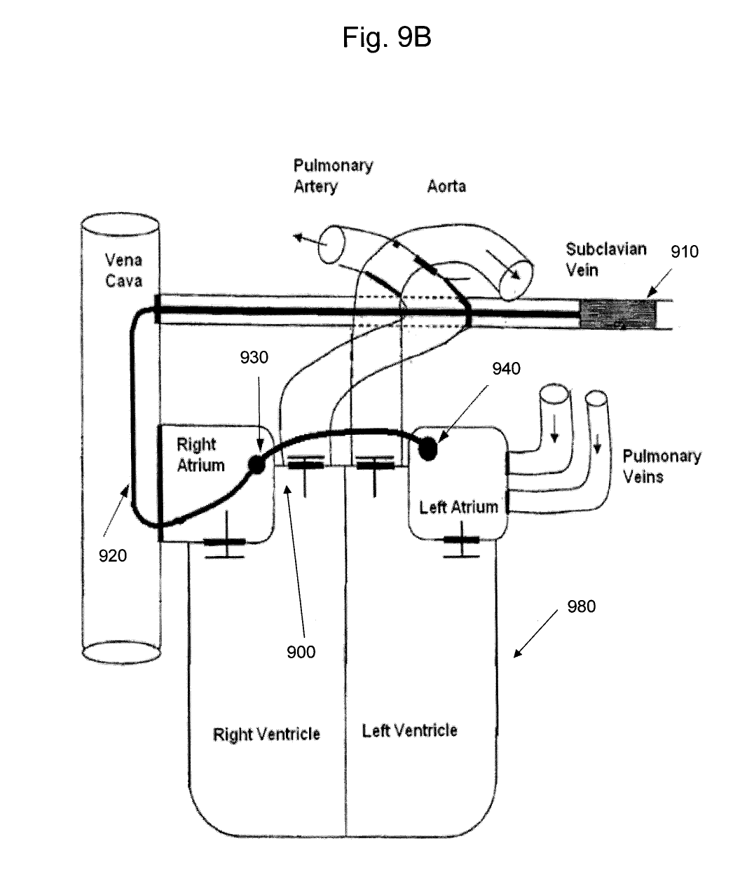

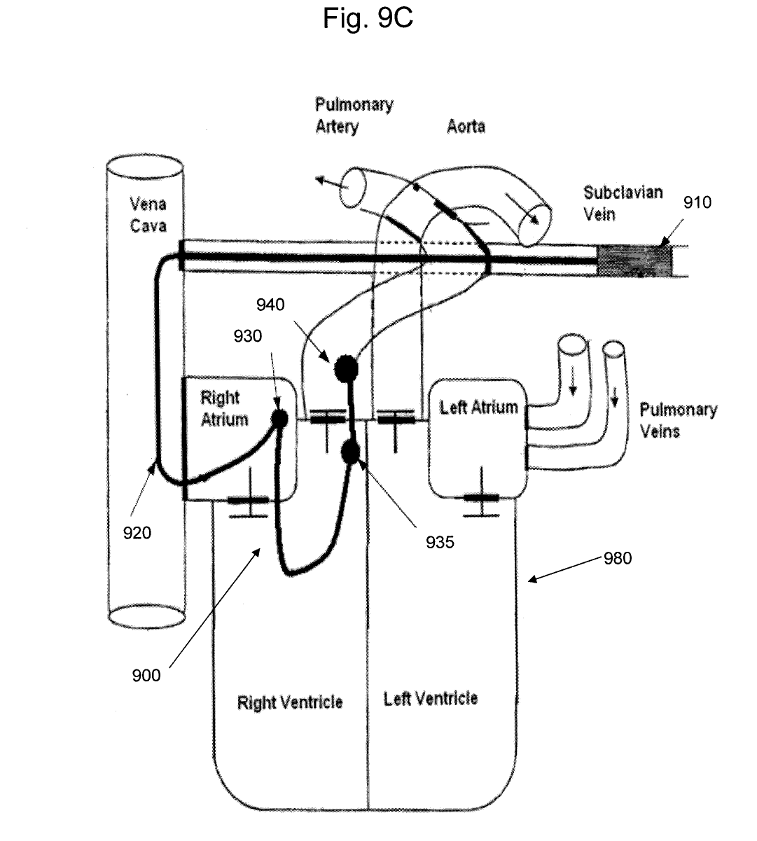

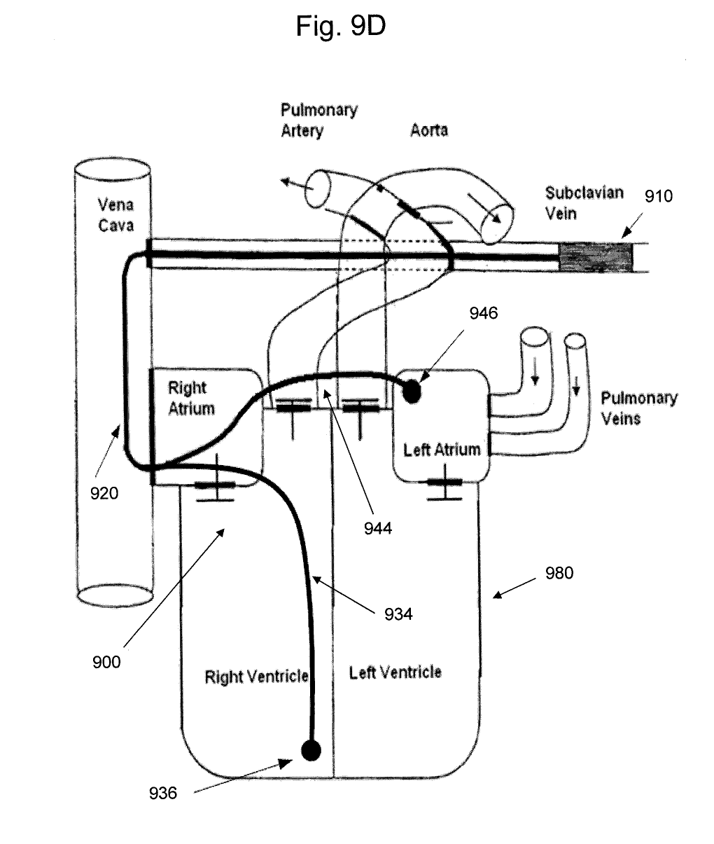

FIG. 9A shows an embodiment of an electrode lead system (900) having a main lead (920) with a central electrode (930) and a distal electrode (940) positioned within a heart (980). The main lead (920) may be connected to a defibrillator (910) located in a side branch of the subclavian vein. In some embodiments, the main lead (920) may enter the heart (980) through the vena cava and into the right atrium, as shown in FIG. 9A. The central electrode (930) may be positioned in the right atrium and the distal electrode (940) may be inserted through the tricuspid valve into the right ventricle. In some embodiments, the distal electrode (940) may be anchored to a wall of the right ventricle near the pulmonary valve. FIG. 9B shows a configuration similar that shown in FIG. 9A, with the exception of the distal electrode (940) being positioned in the left atrium, rather than the right ventricle. In some embodiments, the distal electrode (940) may be inserted into the left atrium through the coronary sinus and near the pulmonary valve.

FIG. 9C shows another embodiment of the electrode lead system (900), wherein the main lead (920) has a first central electrode (930), a second central electrode (935) and a distal electrode (940) positioned within the heart (980). In some embodiments, the defibrillator (910) may be located in a side branch of the subclavian vein. The main lead (920) may enter the heart through the vena cava. The first central electrode (930), which may be a sensing electrode and/or discharging electrode, may be positioned in the right atrium. Furthermore, a central portion of the main lead (920) may be inserted through the tricuspid valve and into the right ventricle so as to position the second central electrode (935) within the right ventricle. In some embodiments, the second central electrode (935) may be anchored to the right ventricle wall near the pulmonary valve. Also as shown in FIG. 9C, a receiving section of the main lead (920) may be inserted through the pulmonary valve and into the pulmonary artery so as to position the distal electrode (940) within the pulmonary artery.

FIG. 9D shows another embodiment of the electrode lead system (900), wherein the main lead (920) is bifurcated and has a first sub-lead (934) with a first electrode (936) and a second sub-lead (944) with a second electrode (946) positioned within the heart (980). In some embodiments, the defibrillator (910) may be located in a side branch of the subclavian vein and the main lead (920) may enter the heart through the vena cava. As shown in FIG. 9D, the first electrode (936) may be positioned within the right ventricle and, in some embodiments, near the apex of the heart (980), which may be particularly advantageous for ventricular defibrillation. The second sub-lead (944) may be inserted through the coronary sinus and into the left atrium near the pulmonary valve so as to position the second electrode (946) in the left atrium.

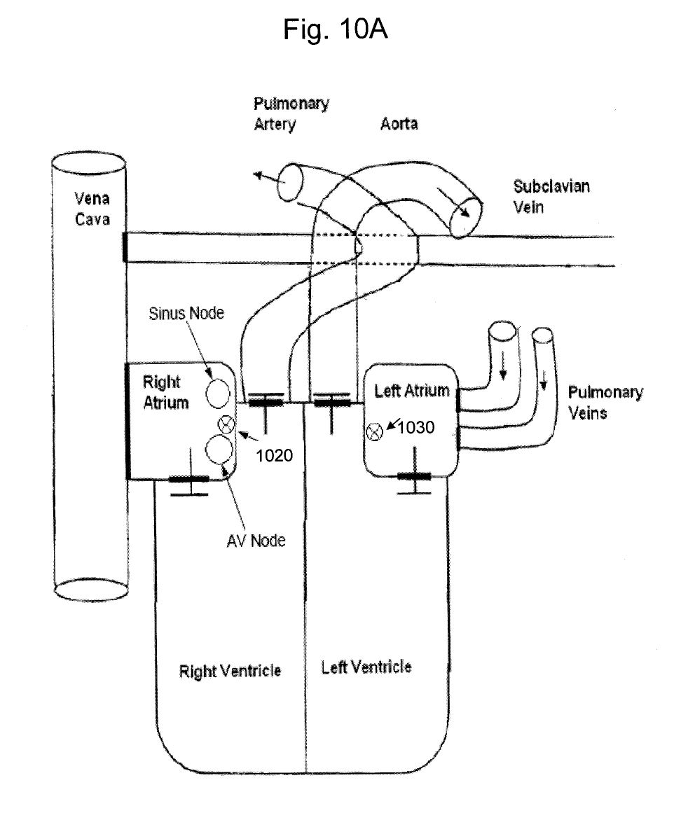

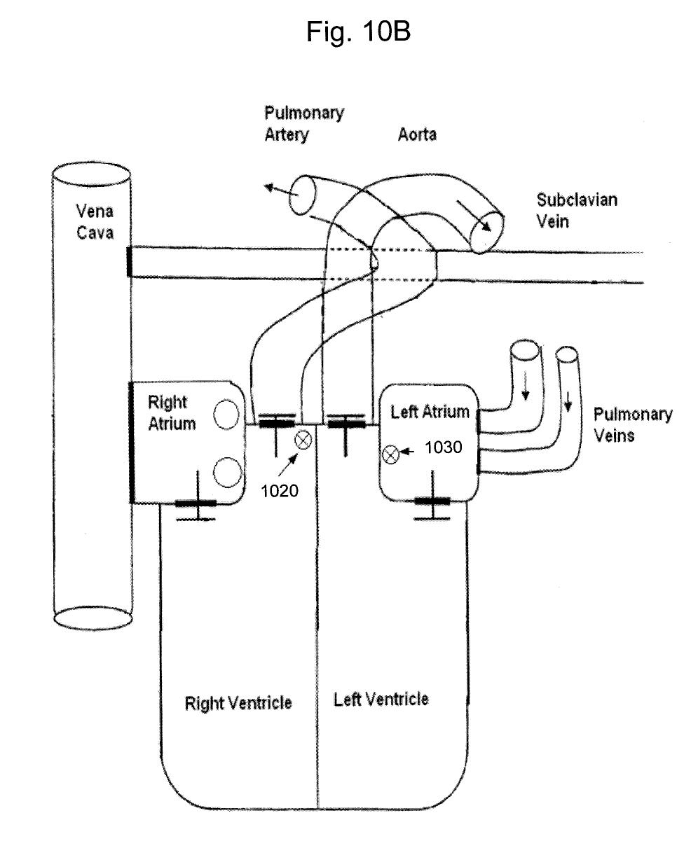

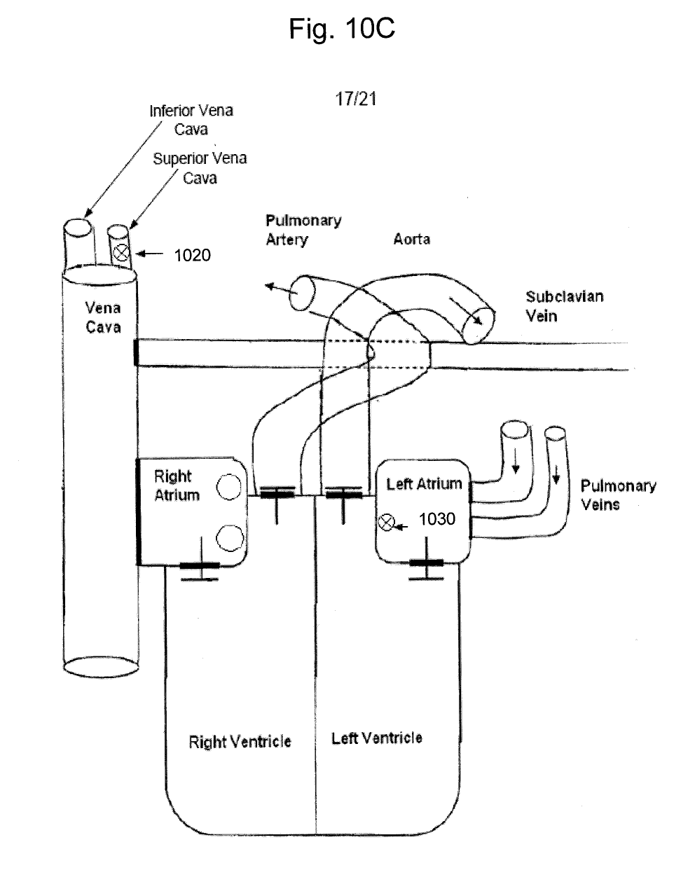

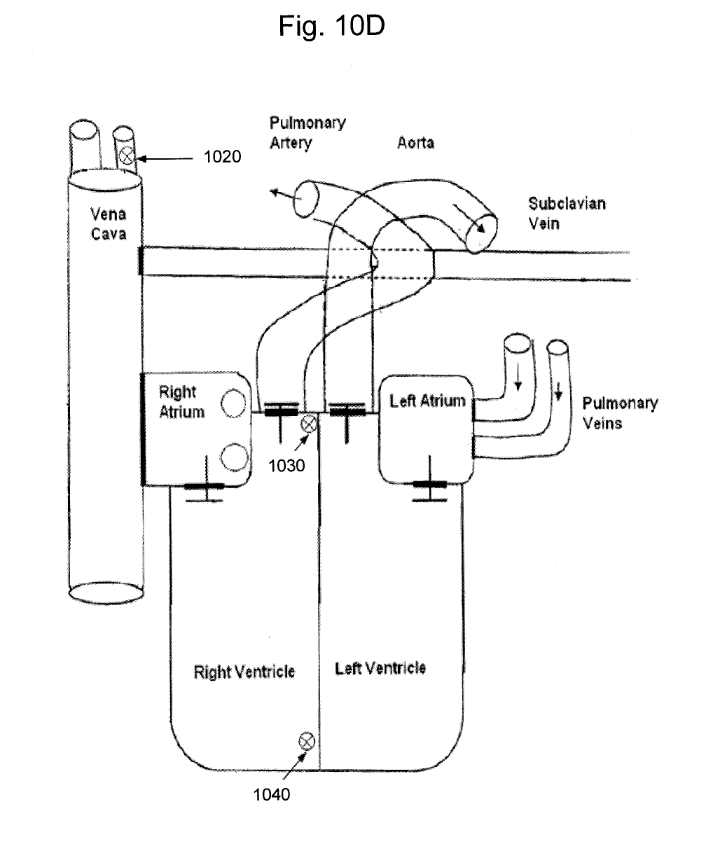

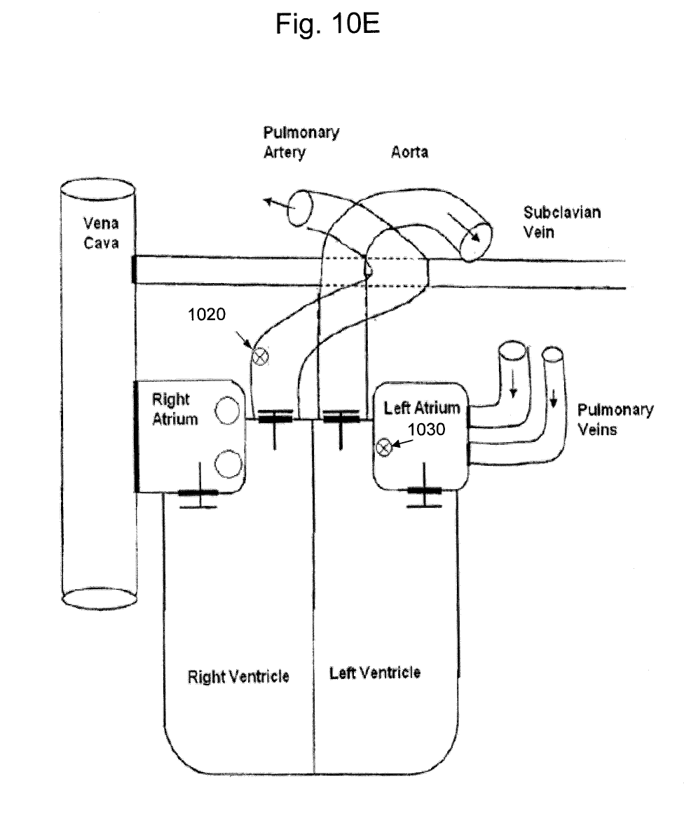

FIGS. 10A-10E show electrode placement configurations according to embodiments of the present disclosure. The electrodes may be sensing electrodes and/or shocking electrodes. The position of each electrode shown in FIGS. 10A-10E may represent the position of an electrode itself or the delivery location of the electrical shock provided by the electrode. For example, an electrode may be positioned in the coronary sinus (or one of the veins surrounding the left atrium) and provide an electrical shock to the left atrium. FIG. 10A shows a first electrode (1020) located near the AV node and the SA node inside the right atrium. The first electrode (1020) may be inserted into the right atrium through the vena cava. A second electrode (1030) may be positioned in the left atrium. In some embodiments, the second electrode (1030) may be positioned at the wall of the left atrium or at the intra-atrial septum, near the pulmonary valve. In some embodiments, the second electrode (1030) may be inserted into the left atrium via the coronary sinus.

FIG. 10B shows a first electrode (1020) located in the right ventricle near the pulmonary valve. The first electrode (1020) may be inserted into the right ventricle through the vena cava and the tricuspid valve. A second electrode (1030) may be positioned in the left atrium. In some embodiments, the second electrode (1030) may be positioned at the wall of the left atrium or at the intra-atrial septum, near the pulmonary valve. In some embodiments, the second electrode (1030) may be inserted into the left atrium via the coronary sinus.

FIG. 10C shows a first electrode (1020) located at the superior vena cava. A second electrode (1030) may be positioned in the left atrium. In some embodiments, the second electrode (1030) may be positioned at the wall of the left atrium or at the intra-atrial septum, near the pulmonary valve. In some embodiments, the second electrode (1030) may be inserted into the left atrium via the coronary sinus.

FIG. 10D shows a first electrode (1020) located at the superior vena cava. A second electrode (1030) may be located in the right ventricle near the pulmonary valve. The second electrode (1030) may be inserted into the right ventricle through the vena cava and the tricuspid valve. A third electrode (1040) may be inserted through the tricuspid valve and positioned located near the apex of the right ventricle. In some embodiments, the third electrode (1040) may be used for sensing and/or ventricular defibrillation and/or pacing.

FIG. 10E shows a first electrode (1020) located in the pulmonary artery and near the pulmonary valve. The first electrode (1020) may be positioned in the pulmonary artery via the right atrium, the tricuspid valve and the right ventricle. Once in the right ventricle, the first electrode (1020) may be moved through the pulmonary valve and into the pulmonary artery. A second electrode (1030) may be positioned in the left atrium. In some embodiments, the second electrode (1030) may be positioned at the wall of the left atrium or at the intra-atrial septum, near the pulmonary valve. In some embodiments, the second electrode (1030) may be inserted into the left atrium via the coronary sinus.

Electrode lead systems according to the subject matter of the present disclosure may include electrodes that are operable to deliver atrial defibrillation pulses, cardiac pacing pulses and/or ventricular defibrillation pulses. One benefit of implementing electrodes that are operable to deliver atrial defibrillation pulses, cardiac pacing pulses and/or ventricular defibrillation pulses is that a defibrillation system primarily configured to, for example, detect atrial fibrillation and deliver atrial defibrillation pulses may also deliver pacing and/or ventricular defibrillation pulses in the event that fibrillation progresses to ventricular fibrillation or ventricular arrhythmia or when the delivered atrial defibrillation shock induces ventricular fibrillation or ventricular arrhythmia. Moreover, providing electrodes configured to deliver cardiac pacing pulses and/or ventricular defibrillation pulses enables atrial defibrillation pulses to be synchronized to the natural or paced ventricular beat. For example, the shock may be synchronized with a patient's cardiac R wave. One benefit of synchronizing the shock and the natural or paced ventricular beat is that the probability that the delivered atrial defibrillation shock would cause ventricular fibrillation or ventricular arrhythmia is reduced.

Some implantable defibrillation system embodiments may include a drug delivering system. The drug delivery system may include a computer-controlled drug pump that is capable of injecting a drug into a right atrium, such as a sedative and/or an anti-arrhythmic drug. The computer controlled drug pump may be controlled by a controller of the defibrillation system. The controller may activate the computer-controlled drug pump prior to the discharge of an electrical pulse. The drug delivery may further reduce the pain and/or discomfort of defibrillation. Furthermore, a drug delivered directly into the heart may take effect more quickly than a drug delivered to the body using an intravenous system.

FIG. 11 shows a flow diagram illustrating a method (1100) for defibrillating an atrium with an implantable defibrillation system. The method (1100) may be also be used to defibrillate a ventricle. The method (1100) includes detecting when an atrium of a heart fibrillates (step 1101), setting electrical pulse parameters (1102), generating an electrical pulse (1103) and discharging an electrical pulse (1104). At step (1101), an implantable defibrillation system may detect when an atrium of a heart is in a fibrillating state. Detecting fibrillation may include receiving sensor signals, such as cardiac functioning signals, from one or more sensors. The sensor signals may be processed to determine whether an atrium is fibrillating. Processing the sensor signals may include signal processing, spatial comparisons or other processes of determining whether a sensor signal indicates that an atrium is fibrillating. At step (1102), electrical pulse parameters are set prior to or upon detection of atrial defibrillation. The electrical pulse parameters may define characteristics, values, boundaries or limitations of the electrical pulse. For example, the electrical pulse parameters may define a discharge voltage and time duration for one or more of the electrical pulses. The electrical pulse parameters may be determined as a function of a distance between a discharge electrode and a receiving electrode and/or voltage difference between a discharge electrode and a receiving electrode. The electrical pulse parameters may define an electrical pulse with a field strength of 100-700 volts/cm and/or a defibrillation voltage of at least 80 volts.

At step (1103) the implantable defibrillation system may generate an electrical pulse in accordance with the electrical pulse parameters. Generating an electrical pulse may include charging a high voltage capacitor with energy, such that an electrical pulse in accordance with the electrical pulse parameters may be discharged from the high voltage capacitor. At step (1104) the implantable defibrillation system may discharge the electrical pulse to an atrium of the heart using a discharge electrode and a receive electrode. Discharging the electrical pulse may include providing energy from a high voltage capacitor to a discharge electrode. Discharging the electrical pulse may also include controlling the discharge.

The method (1100) may further include discharging an electrical pulse train that includes a first electrical pulse and a second electrical pulse. The first electrical pulse and the second electrical pulse may have the same or different discharge voltage, time duration, field strength or a combination thereof. In some embodiments, two or more pulses may be included in the pulse train. The electrical pulses of the pulse train may be monophasic and/or biphasic. The pulse train may include electrical pulses that have or do not have the same polarity as the other electrical pulses in the electrical pulse train. The method (1100) may include other acts. For example, the method (1100) may include implanting a defibrillation system into a heart. In some embodiments, the implantable defibrillation system may be implanted, such that a distance between the discharge electrode and the receiving electrode may be less than 3 centimeters.

In some embodiments, the method (1100) may include one or more notification acts. The method (1100) may be used to notify or communicate with a patient or a control center, such as a hospital or a medical facility. To notify a patient, a notification system may be activated. The notification may be operable to notify a patient of a first electrical pulse before discharging the first electrical pulse to the atrium of the heart. Notifying the patient may include activating a vibration device or acoustic device. To notify a control center, a message may be transmitted to a communication device. The message may be fibrillation message that indicates that an atrium is fibrillating and an electrical pulse has been or will be discharged. Other message may be transmitted to the communication device, such as a location message that indicates the location of the implantable defibrillation system. The location may be determined using a location device, such as a global positioning system. The method (1100) may also include pain reduction acts. The pain reduction acts may include delivering a drug to the heart using the implantable defibrillation system before discharging the first electrical pulse to the atrium of the heart.

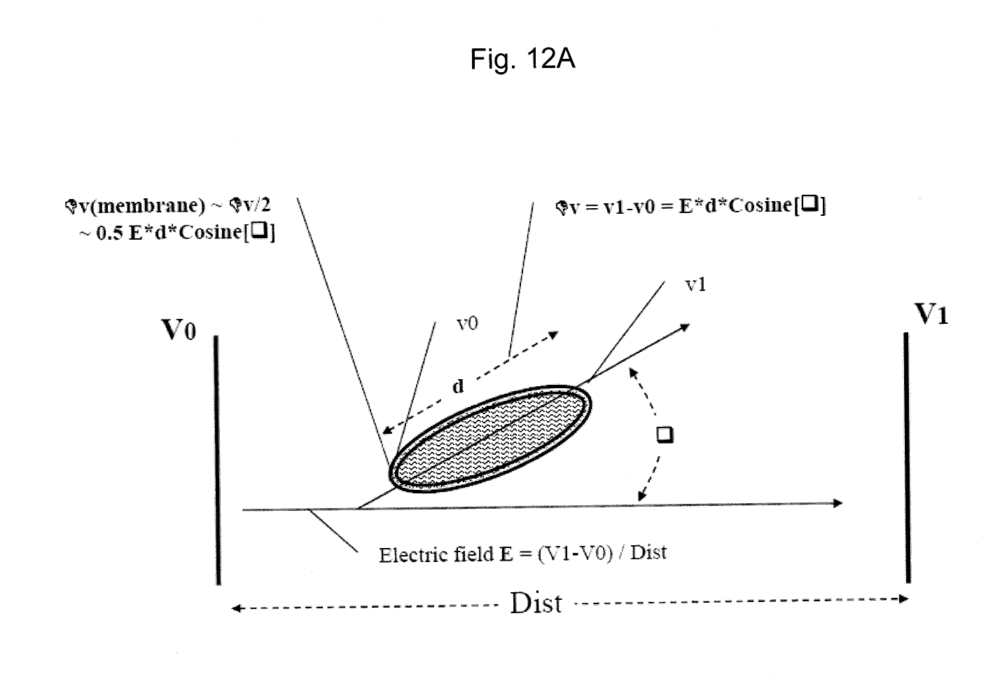

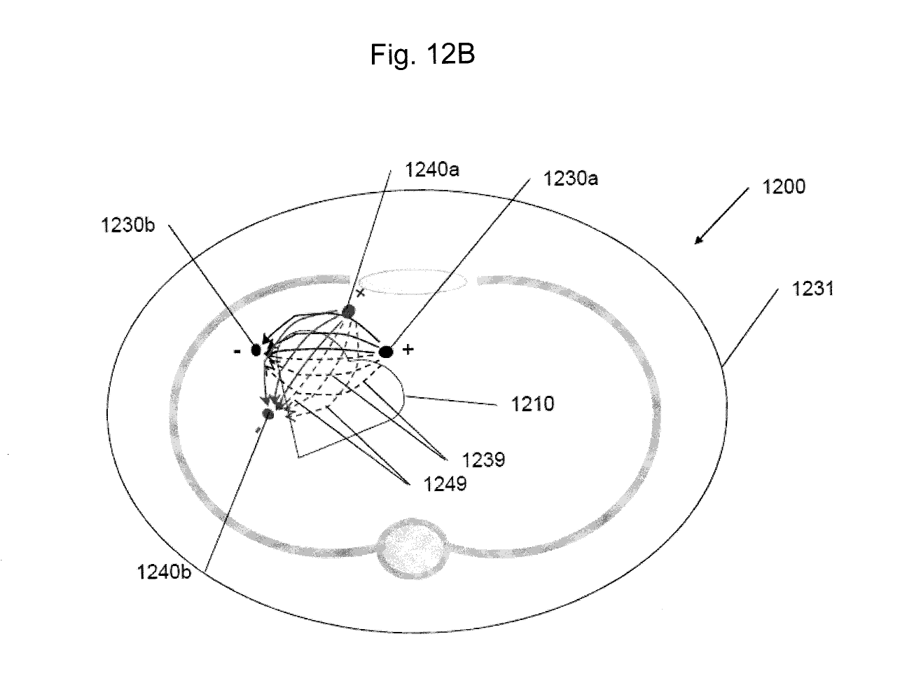

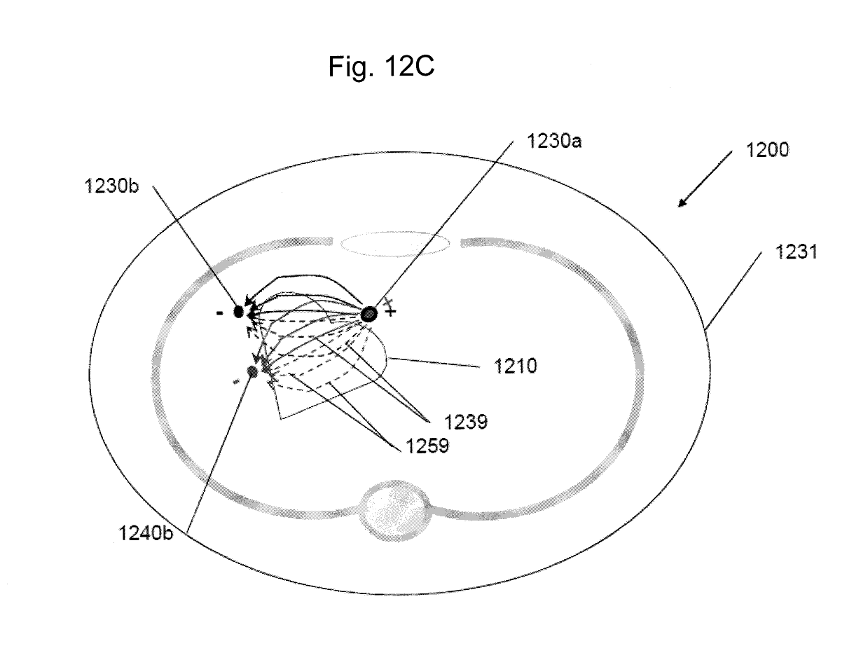

The subject matter of the present disclosure is also directed to embodiments that use more than one electrode pair to defibrillate the heart to increase defibrillation efficiency. More specifically, successful defibrillation involves activating all or at least a majority (e.g., over 90%) of the heart's muscles cells. While electric current generally flows through the somewhat conductive extracellular liquid, cell membranes are generally non-conductive when the cell is not activated. To activate a cell, the defibrillation electric field applied across the membrane of each cell should be above a certain threshold. Heart cells are elongated and may be oriented at an angle with respect to the local direction of the electric field caused by an electrical pulse delivered to the heart. The potential difference becomes a function of both the strength of the electric field and the angle between the longitudinal axis of each cell and the local electric field in its vicinity. For example, referring to FIG. 12A, when an electric field, E, is oriented at an angle, θ, to the longitudinal axis of a cell having a length, d, the induced potential difference on the cell's membrane may be approximated using the equation, Δv (membrane) ˜0.5*E*d*cosine[θ]. To ensure activation, electric field, E, large enough to activate even cells with unfavorable angular orientation may be used.

By using a plurality of electrical pulses having defibrillation electric fields each oriented at different angles increases the probability of successful defibrillation and allows for lower energy electrical pulses to be used, thereby reducing the pain and/or discomfort potentially associated with those pulses. FIGS. 12B and 12C show defibrillation electrical fields delivered to a heart (1210) at different angles using three electrodes or more according to some embodiments of the present disclosure. FIG. 12B shows a cross-section of a patient's torso (1231). Four defibrillation electrodes (1230a), (1230b), (1240a) and (1240b) are positioned in, on and/or around the heart (1210). FIG. 12B shows electrodes (1230a) and (1230b) forming a first pair of electrodes and electrodes (1240a) and (1240b) forming a second pair of electrodes. A defibrillator (not shown) may apply voltage between the first pair of electrodes (1230a) and (1230b) and between the second pair of electrodes (1240a) and (1240b) sequentially. A first voltage, V₁, may be applied between the first pair of electrodes (1230a) and (1230b) to form a first electric field (1239). The electrode (1230a) may be a positive electrode and the electrode (1230b) may be a negative electrode, or vice verse. The first voltage, V₁, may then be removed and a second voltage, V₂, may next be applied between the second pair of electrodes (1240a) and (1240b) to form a second electric field (1249). The electrode (1240a) may be a positive electrode and the electrode (1240b) may be a negative electrode, or vice verse. As shown in FIG. 12B, the resulting field lines of the first electric field (1239) and the second electric field (1249) are not parallel at any point. By positioning the defibrillation electrodes (1230a), (1230b), (1240a) and (1240b) in certain locations in and around the heart (1210), the first electric field (1239) and second electric field (1249) may be at a large angle to each other to sufficiently cover the heart (1210) with defibrillating electrical pulses so as to activate all or at least a majority (e.g., over 90%) of the heart's muscles cells.

FIG. 12C also shows a cross-section of a patient's torso (1231) with three electrodes (1230a), (1230b) and (1240b) positioned in, on and/or around the heart (1210), wherein electrode (1230a) acts as a common electrode. Electrode (1230a) and electrode (1230b) may form a first pair of electrodes and electrodes (1230a) and (1240b) may form a second pair of electrodes. A defibrillator (not shown) may apply first voltage, V_{I}, across the first pair of electrodes (1230a) and (1230b) to form a first electric field (1239). The electrode (1230a) may be a positive electrode and the electrode (1230b) may be a negative electrode, or vice verse. The first voltage, V₁, may then be removed and a second voltage, V₂, may then be applied across the second pair of electrodes (1230a) and (1240b) to form a second electric field (1259). The electrode (1230a) may be a positive electrode and the electrode (1240b) may be a negative electrode, or vice verse. In some embodiments, the common electrode (1230a) may serve as positive electrode for the first voltage, V_{I}, delivered across the first pair of electrodes and a negative electrode for the second voltage, V₂, delivered across the second pair of electrodes. As shown in FIG. 12C, the resulting field lines of the first electric field (1239) and the second electric field (1259) are not parallel at any point. By positioning the defibrillation electrodes (1230a), (1230b) and (1240b) in certain locations in, on and around the heart (1210), the first electric field (1239) and second electric field (1259) may be at a large angle to each other to sufficiently cover the heart (1210) with defibrillating electrical pulses so as to activate all or at least a majority (e.g., over 90%) of the heart's muscles cells.

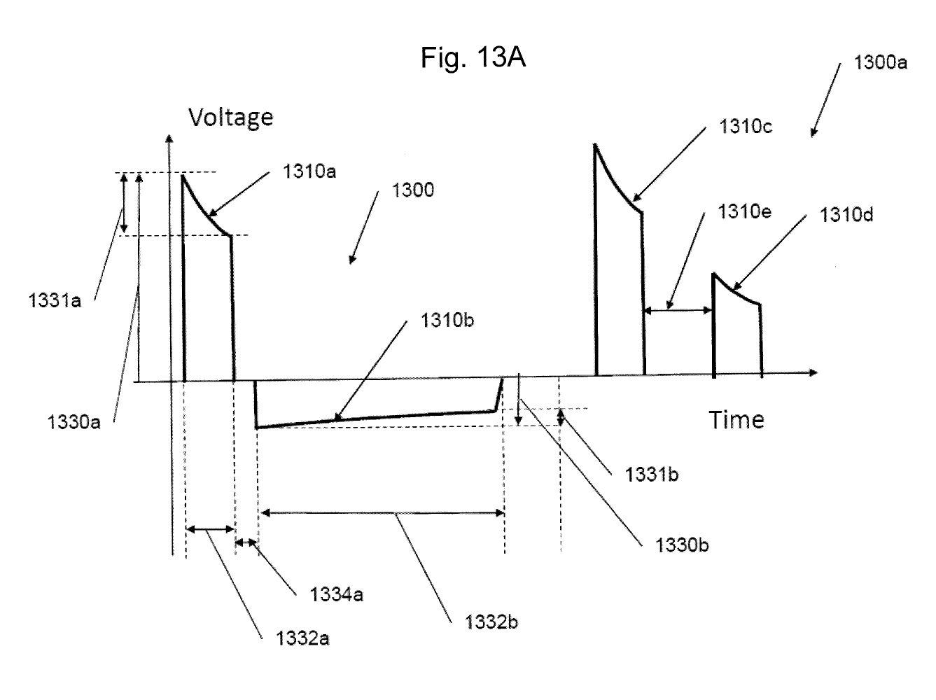

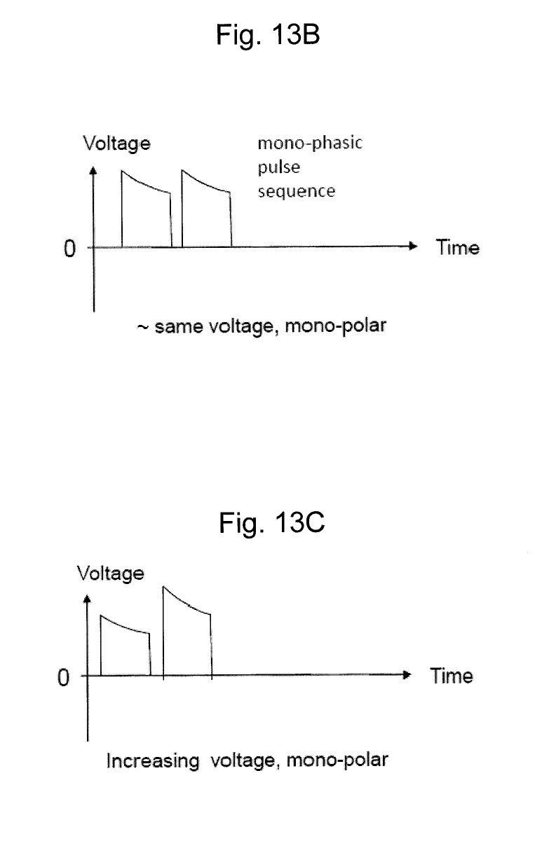

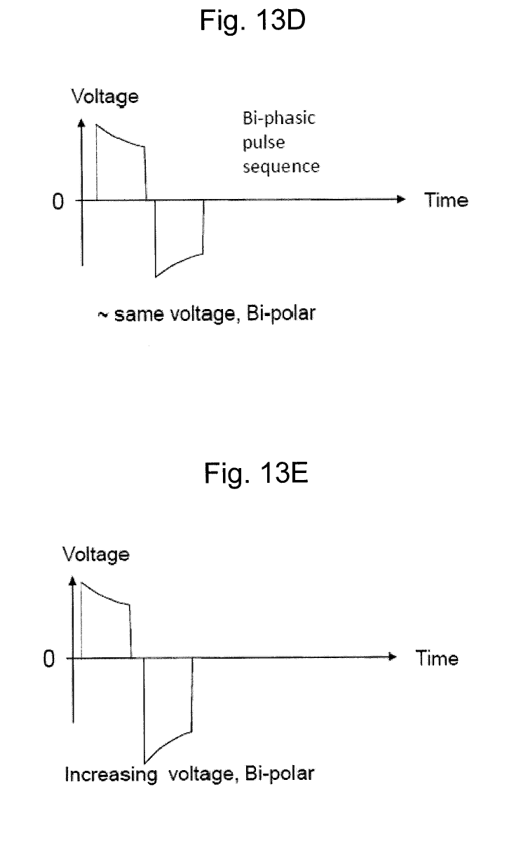

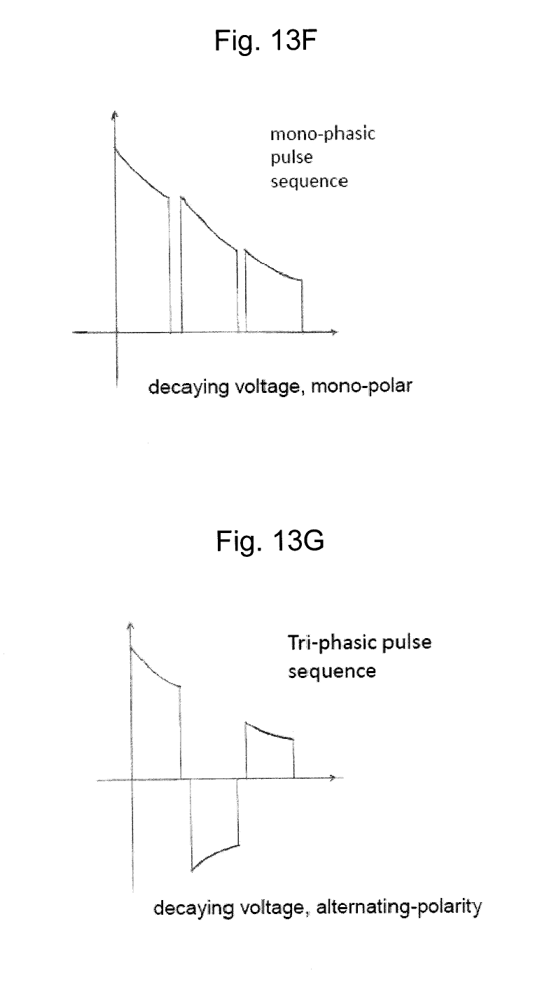

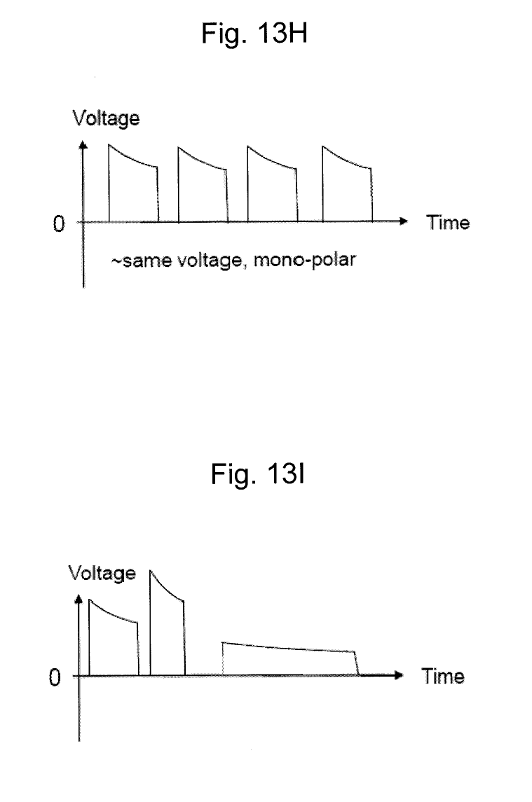

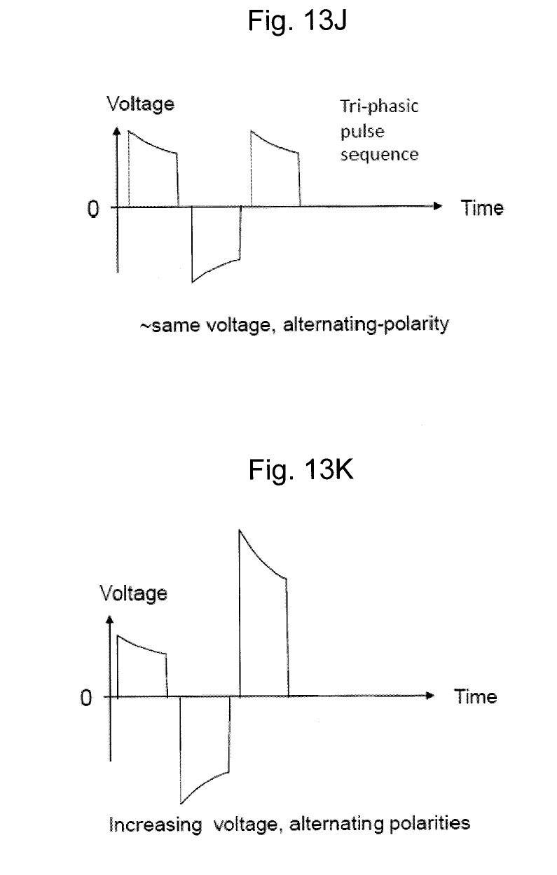

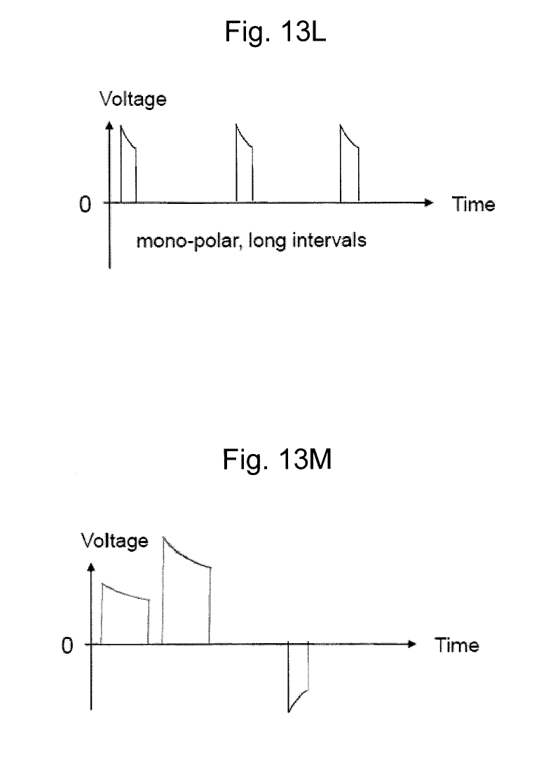

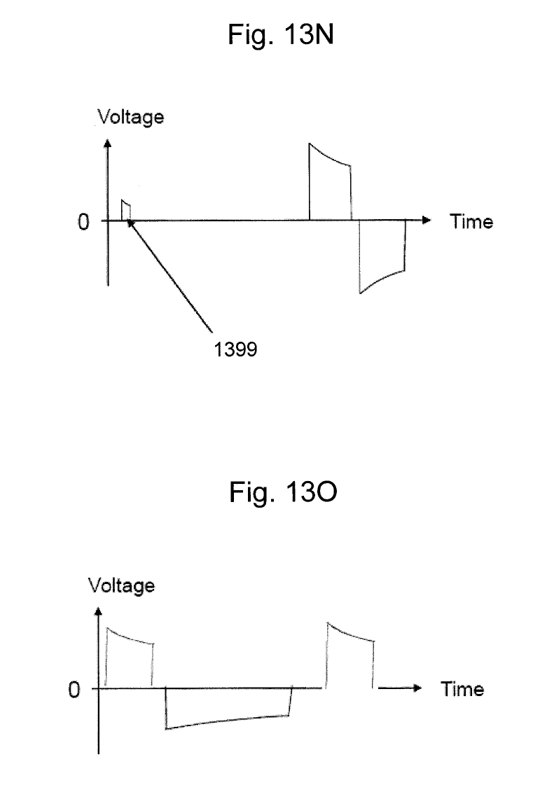

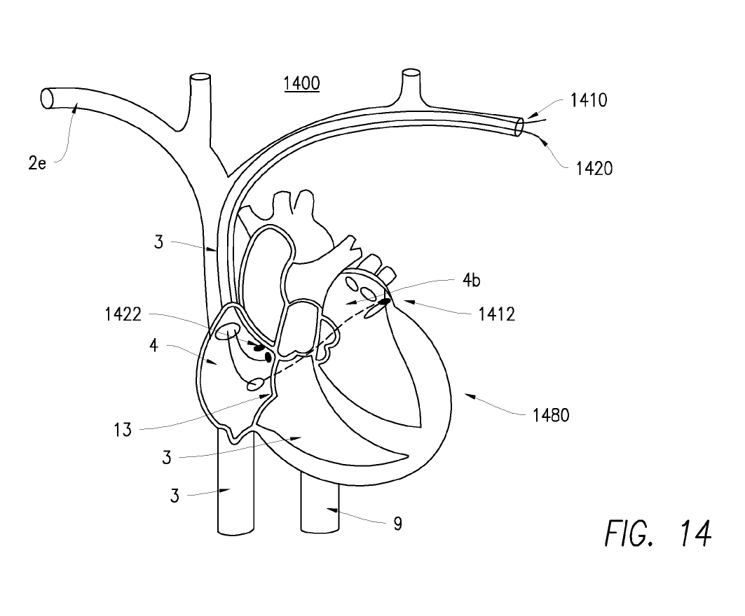

FIGS. 13A-13O show electrical pulse waveform configurations produced by the delivery of electrical pulses to the heart in accordance with the subject matter of the present disclosure. The pulses may be of the same or opposite polarity, of similar, shorter or longer duration and/or of the same, higher or lower voltage. Each pulse may be characterized by initial voltage, duration, voltage drop and/or dwell time (unless the pulse is the last in the pulse train). Other pulse parameters, such as pulse rise time, fall time and ringing, may also be considered and may be influenced by the induction of the pulse circuitry, including without limitation, the electrode leads and the types of switches used in the pulse generation circuitry. When more than two electrodes are used for delivering a pulse, waveforms on different electrodes may differ. A first pulse may be delivered between one pair of electrodes while another pulse or pulses may be delivered between a different pair of electrodes. The different pair of electrodes may have one electrode in common. In some embodiments, three of more electrodes may be used for delivering the same pulse. However, voltage and current may be unevenly divided among the electrodes in a multi-electrode (more than two electrodes) pulse.

In some embodiments, a flexible pulse train generator may be used that allows generation of a pulse train waveform according to the present disclosure. A flexible pulse train generator may be used that provides for the selection of a pulse train waveform from among two or more alternatives. In some embodiments, a pulse train waveform may be tailored to a patient and/or his or her medical condition at the time of the pulse delivery. In addition to known individual variations in the pulse energy required for successful cardioversion, there may be individual variations in the perception of pain or discomfort caused by these pulses. In some embodiments, a default waveform may be used first and, if the default pulse train fails to defibrillate, a second train with different time and energy characteristics may be determined to be more efficacious. In some embodiments, waveforms and other pulse parameters may be tested at a medical facility on a patient and a waveform that efficiently defibrillates, yet results in tolerable discomfort, could be selected and used for future defibrillations.

The use of several pulses of high voltage may be more efficient than delivering the same charge or the same energy in a form of one decaying pulse, since in this waveform, the tissue is subjected to higher voltage throughout the application of the voltage. Increased defibrillation efficiency may advantageous for several reasons. For example, the volume of capacitors used to store electrical energy depends on the maximum possible stored energy. Therefore, reducing the needed energy enables reducing the size of the capacitors and the size of the defibrillator. Similarly, smaller batteries may be used. Additionally, increased defibrillation efficiency may lead to reduced pain or discomfort associated with defibrillation. In addition, the use of two consecutive short pulses with an interval between them could lead to a reduction of pain since the second pulse could be set to stimulate the chest muscle and the nerves at the refractory period of the cells excited by the first pulse, thereby resulting in a reduced chest muscle contraction and nerve response and reduced discomfort and/or pain.

Generating a train of pulses having (i) a second pulse with a voltage larger than the voltage of the first pulse after the second pulse voltage has dropped or (ii) a second pulse with a voltage that is equal to or larger than the voltage of the first pulse, requires using a pulse generator capable of compensating for the voltage drop during the first pulse. A exemplary pulse generator is disclosed in International Patent Application No. PCT/US2011/036828, filed on May 17, 2011 and entitled “Configurable Pulse Generator,” the disclosure of which is incorporated herein by reference.

FIG. 13A shows a pulse train (1300) having a first pulse (1310a) and a second pulse (1310b). The first pulse (1310a) may have an initial voltage (1330a), a voltage drop (1331a), a pulse duration (1332a) and a dwell time (1334a). The voltage drop (1331a) may be determined by the capacitance holding the charge for the first pulse (1310a), the pulse duration and the current that is influenced by the impedance of the tissue of the heart. FIG. 13A also shows a pulse train (1300a) having a first pulse (1310c) and a second pulse (1310d) with a predetermined interval (1310e) between them. The interval (1310e) may be 0-150 milliseconds. In some embodiments, the total length of the pulse train (1300a) should not exceed the individual cardiac refractory period, thus eliminating the possibility that the train would induce ventricular fibrillation as triggered by the R-wave.

FIG. 13B shows a monophasic pulse train having two similar pulses with the same polarity and similar duration and voltage and some pre-programmed interval between, according to some embodiments of the present disclosure. FIG. 13C shows a monophasic pulse train having two pulses with the same polarity and similar duration. In some embodiments, the voltage of the first pulse may be less than the voltage of the second pulse so as to at least partially activate the nerves and muscles in the chest area. In some embodiments, the second pulse may be delivered during the refractory period of these activated muscle fibers and peripheral nerves (and, for safety reasons, also within the refractory period of the contracting heart muscles), is intended to defibrillate the heart. Thus, instead of a single powerful activation of many muscles and peripheral nerves in the chest area, the pulse train will cause only two minor sequential activations, while still having sufficient energy to defibrillate the atria. Such pulse trains may be beneficial in reducing patient's discomfort.

FIG. 13D depicts a biphasic pulse train having two pulses with opposite polarity and similar voltage and duration according to some embodiments of the present disclosure. FIG. 13E shows a biphasic pulse train having two pulses with opposite polarity and similar duration, wherein the voltage of the second pulse may be greater than the voltage of the first pulse. FIG. 13F shows a monophasic pulse train having three pulses with the same polarity and similar duration, wherein the initial voltage of each consecutive pulse is approximately equal to or slightly less than the final voltage of the preceding pulse. FIG. 13G shows a triphasic pulse train having three pulses with alternating polarity and similar duration, wherein the initial voltage of each consecutive pulse is approximately equal to or slightly less than the final voltage of the preceding pulse. FIG. 13H shows a monophasic pulse train having more than three pulses with the same polarity and similar voltage and duration. In some embodiments, at least two of the four pulses may be delivered by a different configuration of electrodes. The first pulse may be delivered between a first pair of electrodes, the second pulse may be delivered between a second pair of electrodes, the third pulse may be delivered between a third pair of electrodes and the fourth pulse may be delivered between a fourth pair of electrodes. FIG. 13I shows a monophasic pulse train having three pulses with the same polarity and dissimilar voltage and duration. FIG. 13J shows a triphasic pulse train having three pulses with alternating polarity and similar voltage and duration. FIG. 13K shows a triphasic pulse train having three pulses with alternating polarity and similar duration, wherein voltage of each consecutive pulse is larger than the voltage of the preceding pulse. FIG. 13L shows a monophasic pulse train having three pulses with the same polarity and similar voltage duration, wherein the dwell time between consecutive pulses is substantially larger than the duration of the pulses. FIG. 13M shows a biphasic pulse train having three pulses with dissimilar voltage and duration, wherein two consecutive pulses have the same polarity which is opposite to the polarity of the third pulse. FIG. 13N shows a pulse train having at least two pulses, wherein the first pulse (1399) in the train is a low energy (e.g., <2J) pulse used to measure the tissue impedance and is used for determining parameters of the following pulse or pulses. In some embodiments, the first pulse (1399) could also be used to desensitize the chest muscles to reduce the discomfort created by the consecutive defibrillation waveform. FIG. 13O shows a triphasic pulse train having three pulses with alternating polarity and dissimilar voltage and duration.

In some embodiments, the pulse trains may be produced such that the net charge delivered to the heart muscle during a defibrillation attempt, is zero. That is, the total charge delivered to the heart during one phase is neutralized by the charge taken from the heart by the portion of the pulse train that is in the opposite polarity. Net charge waveforms need to be at least biphasic. In triphasic or other non-symmetric waveforms, the charge delivered in each phase needs to be calculated and adjusted accordingly. In some embodiments, delivered charge is measured by the defibrillator to adjust the waveform such that zero or near zero net charge would be delivered. For example, the last pulse in the train may be adjusted to compensate for the net charge delivered in the preceding pulses. Zero or near zero net charge train may be used with waveforms which are not mono-polar.

In some embodiments, the use of two or more consecutive short pulses with an interval between them could yield to a reduction of pain since the second pulse could be set to stimulate the chest muscle and the nerves at the refractory period of the cells excited by the first pulse, thus resulting in a reduced chest muscle contraction and nerve response which are expected to be translated to a reduced discomfort and/or pain.

In some embodiments, the use of two or more consecutive short pulses with an interval between them could yield a more efficient defibrillator since the cardiac muscles that were not cardioverted by the first pulse could be cardioverted by the second and/or the consecutive pulses having an equal and/or different polarity and voltage. The whole train though is delivered within the duration of the cardiac refractory period, to avoid induction of ventricular fibrillation.

In some embodiments, where pulses in a train are delivered using at least two different configurations of electrodes, the pulse train may be repeated for each electrode configuration. For example, the pulse train depicted in FIG. 13N may be first applied between a first pair of electrodes. In this case, the first pulse (1399) may serve to measure the impedance between electrodes the two electrodes of the first pair of electrodes. After the completion of the waveform, a second similar train may be applied between a second pair of electrodes. In this case, the first pulse (1399) may serve to measure the impedance between the electrodes of the second pair of electrodes.

In some embodiments, a pulse train for example such as depicted in FIG. 13I may be applied to three electrodes. That is, a first pulse may be applied between a first electrode and the second electrode, a second pulse may be applied between the second electrode and a third electrode and a third pulse may be applied between the third electrode and the first electrode. It should be noted that the above pulse train and electrodes configurations are but examples of such waveforms and electrode configuration possibilities. With different electrode locations, the above pulse train configurations could be used for treating other cardiac arrhythmias, including without limitation, atrial flutter, ventricular tachycardia and ventricular fibrillation. With more electrodes, more electrode configuration exists. Electrode configuration of two pulses in a train may be identical, or identical but with reversed polarity.

FIG. 14 shows an electrode lead system (1400) having a first lead (1410) with a first electrode (1412) and a second lead (1420) with a first electrode (1422) positioned within a heart (1480) according to some embodiments of the present disclosure. The first lead (1410) may enter the heart (1480) through the left subclavian vein so as to position the first electrode (1412) in the right atrium. The second lead (1420) may enter the heart (1480) through the left subclavian vein, extend through the right atrium and into the coronary sinus to position the first electrode (1422) inside the coronary vein on the left side of the heart (1480) proximal to the orifice of the pulmonary veins in the left atrium. In some embodiments, the electric field created by the two electrodes is concentrated in the area of the pulmonary vein orifice—the area most likely to contain the foci of the atrial fibrillation. A strong cardioverting shock in this area would defibrillate the heart while causing minimal effect to the surrounding tissues, where the strength of the electric field will be substantially reduced. Therefore, by delivering the cardioverting shock between two or more electrodes in the region of the pulmonary vein orifice we would defibrillate the atria while causing minimal stimulation to the nearby chest muscles and neural structures. This location of the electrodes will minimize defibrillation threshold and discomfort to the patient undergoing atrial defibrillation.

The embodiments set forth in the foregoing description do not represent all embodiments consistent with the subject matter described herein. It is evident that many alternatives, modifications and variations of such embodiments will be apparent to those skilled in the art. As noted elsewhere, these embodiments have been described for illustrative purposes only and are not intended to be limiting. Thus, other embodiments are possible and are covered by the disclosure, which will be apparent from the teachings contained herein. The breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices; that is, elements from one or another of the disclosed embodiments may be interchangeable with elements from another of the disclosed embodiments. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to any of the disclosed embodiments.

What is claimed is:

1. An implantable defibrillator comprising:
an electrode lead system having at least one lead;
at least one sensor configured to sense a condition of a heart and emit a signal indicative of the condition;
a controller in communication with the at least one sensor, the controller being configured to determine from the signal whether the condition of the heart is one of a state of fibrillation and emit a command signal if the condition is one of a state of fibrillation; and
a voltage generator in communication with the controller and the electrode system, the voltage generator being configured to discharge at least one defibrillation pulse to the electrode system after receiving the command signal,
wherein the at least one defibrillation pulse includes at least one pulse having a voltage greater than 80 volts and a time duration up to 1000 microseconds.

2. The implantable defibrillator of claim 1, wherein the at least one pulse is delivered to an atrium of the heart.

3. The implantable defibrillator of claim 1, wherein the at least one pulse is delivered to a ventricle of the heart.

4. The implantable defibrillator of claim 1, wherein the at least one pulse has an electric field strength between 100 and 700 volts per centimeter.

5. The implantable defibrillator of claim 1, wherein a total amount of energy delivered by the at least one pulse is less than 2 Joules.

6. The implantable defibrillator of claim 1, wherein the time duration of the at least one pulse is between 50 and 600 microseconds.

7. The implantable defibrillator of claim 1, wherein the time duration of the at least one pulse is between 50 and 1000 microseconds.

8. The implantable defibrillator of claim 1, wherein the time duration of the at least one pulse is between 30 and 100 microseconds.

9. The implantable defibrillator of claim 1, wherein the at least one sensor is an electrode of the electrode lead system.

10. The implantable defibrillator of claim 1, wherein the voltage of the at least one pulse is between 80 volts and 3000 volts.

11. The implantable defibrillator of claim 1, wherein the voltage of the at least one pulse is 600 volts or greater.

12. The implantable defibrillator of claim 1, wherein the at least one defibrillation pulse includes at least one pulse having electric field strength between 100 and 700 volts per centimeter, a voltage between 80 and 3000 volts and a time duration between 50 and 1000 microseconds.

13. The implantable defibrillator of claim 1, wherein the at least one pulse is synchronized to the patient's cardiac pulse.

14. The implantable defibrillator of claim 13, wherein the at least one pulse is synchronized to the patient's cardiac R wave.

15. The implantable defibrillator of claim 1, wherein the at least one pulse includes a first pulse and a second pulse, wherein the first pulse has a voltage greater than 80 volts and a time duration less than 1000 microseconds.

16. The implantable defibrillator of claim 15, wherein the second pulse has a voltage greater than 80 volts and a time duration less than 1000 microseconds.

17. The implantable defibrillator of claim 16, wherein the time duration of the second pulse is greater than 100 microseconds.

18. The implantable defibrillator of claim 15, wherein a polarity of the first pulse and a polarity of the second pulse are the same.

19. The implantable defibrillator of claim 9, wherein the first pulse and the second pulse are of opposite polarity.

20. The implantable defibrillator of claim 9, wherein the at least one pulse includes a third pulse.

21. The implantable defibrillator of claim 2, wherein the implantable defibrillator is configured to deliver a defibrillation pulse to a ventricle.

22. The implantable defibrillator of claim 1, wherein a volume of the implantable defibrillator is less than 15 cubic centimeters.

23. The implantable defibrillator of claim 1, wherein the implantable defibrillator is configured to be implanted in a location of the heart selected from the group consisting of the pulmonary vein, the subclavian pocket, a branch of the subclavian vein, the left atrium, the right atrium, the right ventricle, the superior vena cava and the inferior vena cava.

24. The implantable defibrillator of claim 1, wherein the at least one lead includes at least one electrode positioned in a location of the heart selected from the group consisting of the left atrium, the right atrium, the right ventricle, the coronary sinus of the heart, the pulmonary artery, the apex of the right ventricle and the intra-atrial septum of the heart.

25. The implantable defibrillator of claim 24, wherein the at least one electrode is used for sensing.

26. The implantable defibrillator of claim 1, wherein the at least one lead is bifurcated and contains a first sub-lead having at least one electrode positioned in the right atrium and a second sub-lead having at least one electrode positioned in at least one of the right ventricle or the left atrium.

27. The implantable defibrillator of claim 1, wherein the implantable defibrillator is implanted in the right atrium and the at least one lead is a single lead having an electrode positioned in at least one of the right ventricle or the left atrium.

28. The implantable defibrillator of claim 27, wherein the implantable defibrillator acts as an electrode positioned within the right atrium.

29. The implantable defibrillator of claim 1, wherein the at least one lead is a single lead having a first electrode positioned in the right atrium and a second electrode positioned in at least one of the right ventricle or the left atrium.

30. The implantable defibrillator of claim 1, wherein the at least one lead is a single lead having a first electrode positioned in the right atrium, a second electrode positioned in the right ventricle and a third electrode positioned in the pulmonary artery.

31. The implantable defibrillator of claim 1, wherein the at least one lead is bifurcated and contains a first sub-lead having at least one electrode positioned in the left atrium and a second sub-lead having at least one electrode positioned at apex of the right ventricle.

32. The implantable defibrillator of claim 1, wherein the electrode lead system includes a first electrode positioned in the superior vena cava and a second electrode positioned in the left atrium.

33. The implantable defibrillator of claim 1, wherein the electrode lead system includes a first electrode positioned in the superior vena cava and a second electrode positioned in the right ventricle

34. The implantable defibrillator of claim 1, wherein the electrode lead system includes a first electrode positioned in the pulmonary artery and a second electrode positioned in the left atrium.

35. The implantable defibrillator of claim 1, wherein the at least one sensor includes a first sensor and a second sensor in communication with the controller, the first sensor being an electrode for measuring electrical activity of the heart.

36. The implantable defibrillator of claim 35, wherein the second sensor of the at least one sensor includes an electrode for measuring electrical activity of the heart.

37. The implantable defibrillator of claim 35, wherein the controller is configured to determine a location of fibrillation based on signals received by from the first sensor and the second sensor.

38. The implantable defibrillator of claim 37, wherein the controller determines a location of fibrillation based on a plurality of electrocardiogram signals.

39. The implantable defibrillator of claim 35, wherein the second sensor includes a sensing device selected from the group consisting of a microphone, a blood pressure sensor, a thermal sensor, a blood oxygenation sensor, a breathing sensor and an acceleration sensor.

40. The implantable defibrillator of claim 35, wherein the controller is configured to determine a state of atrial fibrillation based on signals communicated from the first sensor and the second sensor.

41. The implantable defibrillator of claim 1, wherein the controller is configured to determine a state of atrial fibrillation based on multi-dimensional signal analysis.

42. The implantable defibrillator of claim 1, wherein the controller is configured to detect a state of ventricle fibrillation and automatically deliver the at least one defibrillation shock when ventricle fibrillation state is detected.

43. The implantable defibrillator of claim 1, the electrode lead system further comprising a first electrode and a second electrode forming a first pair of electrodes and a third electrode and a fourth electrode forming a second pair of electrodes, wherein a first voltage is applied across the first electrode and the second electrode to form a first electric field and a second voltage is applied across the third electrode and the fourth electrode to form a second electric field.

44. The implantable defibrillator of claim 43, wherein the first electric field is at an angle relative to the second electric field.

45. The implantable defibrillator of claim 43, wherein the first voltage applied across the first electrode and the second electrode and the second voltage applied across the third electrode and the fourth electrode are not applied to the heart at the same time.

46. The implantable defibrillator of claim 1, the electrode lead system further comprising a first electrode and a second electrode forming a first pair of electrodes and the first electrode and a third electrode forming a second pair of electrodes, wherein a first voltage is applied across the first electrode and the second electrode to form a first electric field and a second voltage is applied across the first electrode and the third electrode to form a second electric field.

47. The implantable defibrillator of claim 46, wherein the first electric field is at an angle relative to the second electric field.

48. The implantable defibrillator of claim 46, wherein the first voltage applied across the first electrode and the second electrode and the second voltage applied across the first electrode and the third electrode are not applied to the heart at the same time.

49. The implantable defibrillator of claim 1, the at least one pulse further comprising a monophasic pulse train having at least two pulses with substantially the same polarity, duration and voltage.

50. The implantable defibrillator of claim 1, the at least one pulse further comprising a monophasic pulse train having at least a first pulse and a second pulse with substantially the same polarity and voltage, wherein the second pulse has a greater voltage than the first pulse.

51. The implantable defibrillator of claim 1, the at least one pulse further comprising a biphasic pulse train having two pulses with the substantially the same polarity, duration and voltage.

52. The implantable defibrillator of claim 1, the at least one pulse further comprising a biphasic pulse train having at least a first pulse and a second pulse with substantially the same polarity and voltage, wherein the second pulse has a greater voltage than the first pulse.

53. The implantable defibrillator of claim 1, the at least one pulse further comprising a triphasic pulse train having at least three pulses with alternating polarity and substantially the same duration, wherein the initial voltage of each consecution pulse is approximately equal to or slightly less than the final voltage of the preceding pulse.

54. The implantable defibrillator of claim 1, the at least one pulse further comprising a monophasic pulse train having at least three pulses, wherein the initial voltage of each consecution pulse is approximately equal to or slightly less than the final voltage of the preceding pulse.

55. The implantable defibrillator of claim 1, the at least one pulse further comprising a monophasic pulse train having at least four pulses with substantially the same polarity, voltage and duration.

56. The implantable defibrillator of claim 1, the at least one pulse further comprising a monophasic pulse train having at least three pulses with substantially the same polarity and different voltage and duration.

57. The implantable defibrillator of claim 1, the at least one pulse further comprising a triphasic pulse train having at least three pulses with alternating polarity and substantially the same voltage and duration.

58. The implantable defibrillator of claim 1, the at least one pulse further comprising a triphasic pulse train having at least three pulses with alternating polarity and substantially the same duration, wherein the voltage of each consecutive pulse is larger than the voltage of the preceding pulse.

59. The implantable defibrillator of claim 1, the at least one pulse further comprising a monophasic pulse train having at least three pulses with substantially the same polarity, voltage and duration, wherein the dwell time between each pulse is substantially larger than the duration of each pulse.

60. The implantable defibrillator of claim 1, the at least one pulse further comprising a biphasic pulse train having at least a first pulse, a second pulse and a third pulse with different voltages and durations, wherein the first pulse and the second pulse are consecutive and have substantially the same polarity, the polarity of the first pulse and the second pulse being different than the polarity of the third pulse.

61. The implantable defibrillator of claim 1, the at least one pulse further comprising a pulse train having at least a first pulse of less than 2 Joules and used to measure tissue impedance.

62. The implantable defibrillator of claim 1, the at least one pulse further comprising a triphasic pulse train having at least three pulses with alternating polarity and different voltage and duration.

63. The implantable defibrillator of claim 1, the electrode lead system further comprising a first single lead contain an electrode positioned in the inter-atrial septum and a second single lead containing an electrode positioned in the coronary vein.

64. A heart defibrillation system comprising:
a defibrillator configured to be implanted in a patient, the defibrillator comprising:
an electrode lead system having at least one lead;
at least one sensor configured to sense a condition of a heart and emit a signal indicative of the condition;
a controller in communication with the at least one sensor, the controller being configured to determine from the signal whether the condition of the heart is one of a state of fibrillation and emit a command signal if the condition is one of a state of fibrillation;
a voltage generator in communication with the controller and the electrode system, the voltage generator being configured to discharge at least one defibrillation pulse to the electrode system after receiving the command signal, wherein the at least one defibrillation pulse includes at least one pulse having a voltage greater than 80 volts and a time duration up to 1000 microseconds; and
a communication device disposed outside of the patient configured to communicate with the defibrillator.

65. The heart defibrillation system of claim 64, wherein the communication device includes notification circuitry configured to notify the patient that fibrillation was detected.

66. The heart defibrillation system of claim 65, wherein the notification circuitry is configured to notify the patient that fibrillation was detected and to instruct the patient to be prepared for an defibrillation shock.

67. The heart defibrillation system of claim 65, wherein the notification circuitry is configured to instruct the patient to seek medical treatment in a medical center.

68. The heart defibrillation system of claim 65, wherein notification circuitry is configured to notify the patient of a worsening cardiac condition.

69. The heart defibrillation system of claim 64, wherein the communication device is configured to initiate an atrial defibrillation shock.

70. The heart defibrillation system of claim 64, wherein the communication device is configured to notify a medical facility of a cardiac condition of the patient.

71. The heart defibrillation system of claim 70, wherein the communication device includes location determination circuitry configured to determine a location of the patient and is configured to communicate the determined location to a medical center.

72. The heart defibrillation system of claim 70, wherein the communication device is configured for bi-directional communication with the implantable defibrillator over a short range wireless communication link, and is configured for bi-directional communication with the medical center over a long-range wireless communication link.

73. The heart defibrillation system of claim 71, wherein the long-range wireless communication link is cellular communication link.

74. The heart defibrillation system of claim 71, wherein the communication device is a mobile phone.

75. The heart defibrillation system of claim 37, wherein a message communicated over the long-range communication link is a message selected from the group consisting of a synthesized voice announcement, a pre-recorded voice announcement, a short message service, a multimedia message service and electronic mail.

76. A method for defibrillating a heart with an implantable defibrillator, the method comprising:
detecting when a condition of fibrillation within the heart;
configuring at least one electrical pulse parameter to define an electrical pulse having a voltage between 80 and 3000 volts and a duration between 30 and 1000 microseconds;
generating a first electrical pulse in accordance with the at least one electrical pulse parameter; and
discharging the first electrical pulse to the heart using an electrode lead system having at least one pair of electrodes positioned in or around the heart.

77. The method as claimed in claim 76, wherein discharging the first electrical pulse includes generating an electric field strength of between 100 to 700 volts per centimeter across the at least one pair of electrodes.

78. The method as claimed in claim 76, the method further comprising transmitting a fibrillation message to a medical center when the atrium in the heart fibrillates.

79. The method as claimed in claim 78, comprising determining a location of the implantable heart defibrillator using location determination circuitry, the location being included in a fibrillation message that enables the medical center to determine the location of the implantable defibrillation system.

80. The method as claimed in claim 78, comprising delivering a drug to the heart using the implantable heart defibrillator before discharging the first electrical pulse to the atrium of the heart.

81. The method as claimed in claim 78, comprising activating a notification circuitry configured to notify a patient of the first electrical pulse before discharging the first electrical pulse to the atrium of the heart.

82. A method of reducing pain while defibrillating an atrium of a human heart, the method comprising:
delivering at least one pulse to the atrium having a voltage greater than 600 volts and a time duration between 50 and 600 microseconds.

83. A method of reducing pain while defibrillating a ventricle of a human heart, the method comprising:
detecting a condition of ventricular fibrillation within the heart using an implantable defibrillator;
configuring at least one electrical pulse parameter to define an electrical pulse having a voltage between 80 and 3000 volts and a duration of 50 to 1000 microseconds;
generating a first electrical pulse in accordance with the at least one electrical pulse parameter; and
discharging the first electrical pulse from the implantable defibrillator to the heart using an electrode lead system having at least one pair of electrodes positioned in or around the heart,
wherein a total amount of energy delivered by the first electrical pulse is less than 2 Joules.

84. The method of claim 83, wherein the first electrical pulse is monophasic.

85. The method of claim 83, wherein the first electrical pulse is biphasic.

86. The method of claim 83, wherein the implantable defibrillator acts as an electrode of the electrode lead system.
